(19) United States
(12) Patent Application Publication
(10) Pub. No.: US 2023/0049309 A1
(43) Pub. Date: Feb. 16, 2023

(54) CATHETER DEVICE FOR RELEASING PHARMACEUTICALLY ACTIVE COMPOUNDS OVER AN EXTENDED PERIOD

(71) Applicant: CIRLO GMBH, Wolfratshausen (DE)

(72) Inventors: Sabine Bauer, Wolfratshausen (DE); Thomas Ischinger, Munich (DE)

(73) Assignee: CIRLO GMBH, Wolfratshausen (DE)

(21) Appl. No.: 17/787,128

(22) PCT Filed: Dec. 16, 2020
(86) PCT No.: PCT/EP2020/086532

§ 371 Date: Jun. 17, 2022

(30) Foreign Application Priority Data
Dec. 17, 2019 (GB) 1918670.9

(51) Int. Cl. A61L 29/16; A61L 29/14

(52) U.S. Cl. CPC A61L 29/16; A61L 29/146; A61L 2300/414

(57) ABSTRACT

The present invention relates to a catheter device comprising at least one pharmaceutically active compound, wherein the device is capable of releasing said pharmaceutically active compound such as angiogenesis promoting factors, inhibitors of an angiogenesis inhibiting factor or antibiotics over an extended period. It is preferred that the device is tubular and longitudinally extending device assembly with an intraluminal and an extraluminal segment and a proximal and a distal end, wherein the intraluminal segment comprises at least one reversibly expandable portion. Also envisaged is a catheter device for use in preventing a slow-healing or non-healing wound, diabetic foot or intoxication with bacterial toxins, or for treating or preventing bacterial or virus infections and/or medical complications during transplantation.

### FIELD OF THE INVENTION

The present invention relates to a catheter device comprising at least one pharmaceutically active compound, wherein the device is capable of releasing said pharmaceutically active compound such as angiogenesis promoting factors, inhibitors of an angiogenesis inhibiting factor or antibiotics over an extended period. It is preferred that the device is tubular and longitudinally extending device assembly with an intraluminal and an extraluminal segment and a proximal and a distal end, wherein the intraluminal segment comprises at least one reversibly expandable portion. Also envisaged is a catheter device for use in preventing a slow-healing or non-healing wound, diabetic foot or intoxication with bacterial toxins, or for treating or preventing bacterial or virus infections and/or medical complications during transplantation.

### BACKGROUND OF THE INVENTION

It is estimated that there will be more than 400 million diabetics worldwide by 2025, with the greatest increases in Asia, Africa, and South America. It is expected that these patients will develop foot ulcers during their lifetime (Singh et al., JAMA 2005; 293:217-228). However, these staggering figures account for only one of the wounds that come to the attention of the woundologist. In the United States, the National Institutes of Health estimates that 3% of the population over the age of 65 will have a wound at any one time (Sen et al., Wound Repair Regen 2009; 17:763-771). In resource-poor regions of the world, an even greater incidence is expected due to traumatic injuries and ulcers endemic to other regions of the world. India, for example, is estimated to have one of the highest incidences of traumatic wounds (10.5 per 1,000). In 30 countries in Africa and other tropical regions, Mycobacterium ulcerans infection leads to the development of the difficult-to-heal Burili ulcer.

However, there is wide variation in the reported prevalence and incidence of chronic wounds worldwide and within each care setting. The most prevalent wounds are venous leg ulcers LU), pressure ulcers (PU) and diabetic foot ulcers (DFU) in people aged>60. A percentage of wounds may not heal completely for a year or more, and this places a significant burden on health-care systems and economies. In the context of this consensus statement, complete healing means full epithelial resurfacing and discharge, or transition to patient-management strategies to prevent recurrence.

In 2010, the World Health Organization (WHO) recognized the need to address the worldwide problem of wounds. Under the aegis of the WHO, the World Alliance for Wound and Lymphedema Care (WAWLC) was formed to provide education and assist in the development of centers of excellence in wound healing.

According to the WHO open injuries have a potential for serious bacterial wound infections, including gas gangrene and tetanus, and these in turn may lead to long term disabilities, chronic wound or bone infection, and death. Wound infection is particularly of concern when injured patients present late for definitive care, or in disasters where large numbers of injured survivors exceed available trauma care capacity. Appropriate management of injuries is considered important to reduce the likelihood of wound infections. The WHO has therefore established wound healing protocols which comprise, inter alia, steps like avoiding the closure of infected wounds, performing wound toilet and surgical debridement, cleaning wounds that are more than 6 hours old, restoring breathing and blood circulation as soon as possible after injury and giving antibiotic prophylaxis to victims with deep wounds and other indications. However, antibiotics typically do not reach the source of the wound infection. Antibiotics instead only reach the area around the wound.

One important factor for wound healing is hence angiogenesis and sufficient blood supply. During wound healing, angiogenic capillary sprouts typically invade the fibrin/fibronectin-rich wound clot and within a few days organize into a microvascular network throughout the granulation tissue. As collagen accumulates in the granulation tissue to produce scar, the density of blood vessels diminishes. A dynamic interaction occurs among endothelial cells, angiogenic cytokines, such as FGF, VEGF, TGF-beta, angiopoietin, and mast cell tryptase, and the extracellular matrix (ECM) environment. Specific endothelial cell ECM receptors are critical for these morphogenetic changes in blood vessels during wound repair.

For an effective treatment of slow or non-healing wounds there is thus a requirement for a concerted angiogenetic and antimicrobial approach. The presently provided strategies, such as integrated patient care, patient management approaches and the combat of biofilms have provided some effects but are still not capable of providing a substantial solution to the underlying problem.

There is therefore a need for an alternative therapeutic approach and suitable implementations approach, which allows to effectively treat this type of highly dangerous conditions.

### OBJECTS AND SUMMARY OF THE INVENTION

The present invention addresses this need and provides a catheter device comprising at least one pharmaceutically active compound, wherein the device is capable of releasing said pharmaceutically active compound over an extended period.

The present inventors have surprisingly found that the use of a catheter device which is capable of releasing an angiogenesis promoting factor and/or an inhibitor of an angiogenesis inhibiting factor over an extended period can contribute to a significant improvement of healing processes in non-healing wounds or slow healing wounds. The catheter device is further designed to release additional pharmaceutically active compound such as antibiotics. The combined provision of angiogenesis promoting factors and of anti-microbial compounds has been found to be highly efficient in the treatment and management of slow or non-healing wounds. A further advantage of the inventive approach is the efficient neutralization of bacterial toxins via interaction with suitable binding elements.

An additional advantage of the inventive device is its perioperative and post-operative usability for an efficient wound healing. By releasing the mentioned pharmaceutically active compounds increases the success rate of the treatment. Moreover, the local and selective use in a chosen target vessel advantageously allows the device to be inserted into the body by a routine minimally invasive catheter procedure, for example after puncture of the target vessel or a vessel leading to the target. Also the replacement of certain components of the device, in particular membranes and associated micro- and macroreservoirs allow for a very efficient use over a prolonged period of time. The present invention's approach thus significantly improves the therapy and prognosis of patients, in particular of patients being affected by non-healing wounds or in in danger of developing such wounds.

Vascular stent and filter technology, which resembles the presently claimed approach on a mechanistic level, is well established. A stent usually is a permanent implant into a diseased and obstructed artery or vein in order to improve blood flow and maintain patency of the formerly obstructed vessel. Stents may, for example, be balloon expandable or self-expandable. Balloon expandable stents are typically made of plastically deformable material such as 316 L steel or cobalt-chrome alloys, and are mounted on a deflated balloon, positioned in the target zone and expanded by inflation of the balloon. Self-expandable stents are usually, but not exclusively, made of a nitinol mesh, i.e. an elastically deformable, memory metal, which is held constraint on a catheter by an outer constraining mechanism and is released in the target organ by withdrawal of the constraining tube. The self-expanding stent then takes its predetermined shape via the memory metal effect.

In addition, there are examples of capturing devices such as the Filterwire (Boston Scientific) or Spider FX (Covidien) device (see also FIG. 11), which are essentially filters resembling umbrellas on a wire with uniform pores of typically 80 μm-110 μm in diameter. Their purpose is the capture of macroscopically visible, large size debris during an interventional procedure. At termination of the procedure the filters are withdrawn. Also known in the art is the so called vena cava filter (Cook, Crux Biomedical), which is a crude filter device that is released in the Vena cava in patients with extremely high risk of spontaneous venous thromboembolism. The purpose of this filter is to trap large masses of thrombotic material in order to avoid pulmonary embolism. While it is intended for permanent implantation, it may be retrieved by lasso or other known catheter techniques as long as it is not overgrown by tissue or not perforated into the vessel wall. Recently, a non permanent vena cava embolic filter has been introduced for the prevention of pulmonary embolism in high risk medical situations in critically ill patients, i.e. during trauma surgery and when anticoagulation is contraindicated (Angel catheter, Bio2Medical).

There is, however, no prior art disclosure of a dedicated catheter device assembly, which is specially designed for non-permanent use in specific parts of the vascular system and for being capable of releasing certain pharmacologically active compounds over a longer period of time and additionally featuring, for example, inter alia self-expansion and antimigratory mechanical properties, and clogging resistance according to the present invention. Thus, while resembling some aspects of traditional stents and conventional embolic filters, the catheter device of the present invention advantageously transforms some of the physical features of traditional stent and emboli (blood clots) preventing filter technology into pharmaceutical composition-like devices which are capable of fulfilling completely different purposes such as substantial wound healing.

Another advantage of the presently envisaged device is that the device is designed to maintain blood flow even through microscopically small filter structures by creating areas with unhindered flow within or adjacent to filter membranes in order to minimize reduction or stasis of flow or thrombosis. This approach is in clear contrast to devices known in the art, which are typically intended to completely cover the cross-section of a vessel in order to provide full embolic protection.

In a preferred embodiment of the catheter device said pharmaceutically active compound is an angiogenesis promoting factor and/or an inhibitor of an angiogenesis inhibiting factor.

It is particularly preferred that said angiogenesis promoting factor is a promoting chemokine, a fibroblast growth factor, hepatocyte growth factor (HGF), a hypoxia-inducible factor, a platelet-derived growth factor, a transforming growth factor beta, or a vascular endothelial growth factor.

In a further preferred embodiment, said promoting chemokine is CXC-1, CXC-2, CXC-3, CXC-5, CXC-6, CXC-7, or CXC-8.

In another preferred embodiment, said fibroblast growth factor is FGF-1 or FGF-2.

In a further preferred embodiment said hypoxia-inducible factor is HIF-1, HIF-2 or HIF-3.

In yet another preferred embodiment said platelet-derived growth factor is PDGF-A, PDGF-B, PDGF-C or PDGF-D.

In a further preferred embodiment said transforming growth factor beta is TGF-beta-1, TGFbeta-2 or TGFbeta-3.

It is further preferred that the vascular endothelial growth factor is VEGF-A, VEGF-B, VEGF-C, VEGF-D or PIGF.

In another preferred embodiment said angiogenesis inhibiting factor is an angiopoietin, angiostatin, an inhibiting chemokine, endostatin, an interferon, pigment epithelium-derived factor (PEDF) or a thrombospondin.

In yet another preferred embodiment, said angiopoietin is Ang-1 or Ang-2.

It is further preferred that said inhibiting chemokine is CXC-4, CXC-9, CXC-10, CXC-11, CXC-12, or CXC-14.

It is further preferred that said interferon is INF-alpha, INF-beta or INF-gamma.

According to a further preferred embodiment, said thrombospondin is TSP-1, TSP-2, TSP-3, TSP-4 or TSP-5.

In a further embodiment, the device according to the present invention additionally comprises a further pharmaceutically active compound of the group of antibiotics, anticoagulants and/or analgesics and/or serotonin and/or divalent ions such as Ca²⁺ or Mg²⁺.

In a particularly preferred embodiment of the present invention said antibiotic is penicillin such as amoxicillin, ampicillin, oxacillin or dicloxacillin; a tetracycline such as demeclocycline, doxycycline, eravacycline, minocycline or omadacycline; a cephalosporin such as cefaclor, cefotaxime, ceftazidime, cefuroxime; a quinolone such as ciprofloxacin, levofloxacin or moxifloxacin; a lincomycin such as clindamycin, or lincomycin; a sulphonamide such as sulfamethoxazole, trimethoprim or sulfasalazine; a glycopeptide antibiotic such as dalbavancin, ortavancin, telavancin or vancomycin; an aminoglycoside such as gentamicin, tobramycin, or amikacin; an ansamycine such as rifampicin, rifamycin B, rifamycin SV, rifabutin, rifapentine, or rifamixin; fosfomycin; fusidic acid; linezolid; or a carbapenem such as imipenem, cilastatin, merpoenem, dorpenem or ertapenem, or minocylin.

In a further aspect the present invention relates to catheter device as defined herein comprising at least one elements which is capable of binding to or capturing a virus, preferably an ACE2 receptor or a sub-portion thereof, a human ACE2 (hACE2) protein or a truncated version thereof, or sub-portion thereof, a Coronaviridae spike protein such as a SARS-CoV-2 spike protein or a sub-portion thereof, or a proprotein convertase furin.

In yet another preferred embodiment, the device is a tubular and longitudinally extending device assembly with an intraluminal and an extraluminal segment and a proximal and a distal end.

In particularly preferred embodiment the intraluminal segment comprises at least one reversibly expandable portion.

It is further preferred that the expandable portion extends at least 2 mm longitudinally.

In yet another embodiment, the expandable portion is permeable for a bodily fluid, preferably for blood.

In a further preferred embodiment the expandable portion is capable of increasing the surface area of the intraluminal segment at least two fold over the length of the expandable portion.

In yet another preferred embodiment, the device according to the present invention comprises the pharmaceutically active compound(s) in one or more compartments.

It is preferred that said compartments are capable of releasing said pharmaceutically active compound(s) over an extended period and/or can be controlled externally, preferably via a handheld device.

In a specific embodiment said extended period is a period of about 7 days to 3 months.

In a further embodiment said compartments are provided in the at least one expandable portion, which is liquid permeable and capable of releasing said pharmaceutically active compound(s) into bodily fluids or target tissues.

In a further preferred embodiment the compartment is a drug containing surface area or macro-reservoir.

In yet another preferred embodiment said drug containing surface area comprises a porous surface, or comprises one or more drug reservoirs such as micro-reservoir.

In yet another preferred embodiment the reservoir(s) is/are imbedded within a membrane, preferably a porous membrane, or within the porous surface.

In yet another preferred embodiment the at least one expandable portion is a porous balloon.

In a further preferred embodiment the porous balloon in its expanded state is at least one third smaller in diameter than the diameter of the luminal target organ.

In an additional embodiment the porosity of the balloon permits selective fluid permeability and wherein said permeability permits interaction with surrounding environments by controlled release of pharmaceutically active compound(s) from the balloon and attraction of surrounding fluid elements to the balloon.

In yet another preferred embodiment of the device, multiple expandable portions are arranged along the longitudinal extension of the intraluminal device in a tandem position and all expandable portions permit flow of a bodily fluid around or through the expandable portions.

In yet another preferred embodiment the at least one expandable portion is in fluid connection via a tubular channel with the proximal end of the extraluminal segment.

In a further preferred embodiment the longitudinally extending tubular device comprises at least one wire channel extending from the distal tip of the intraluminal segment through at least a portion of the extraluminal segment.

In a further preferred embodiment the expandable portions are a combination of a scaffold and balloon.

Is further preferred that the at least one expandable active portion is detachable for anchoring within the luminal target organ.

In yet another preferred embodiment the reversible anchoring mechanism is comprised within the detachable device.

In yet another preferred embodiment the active expandable portion is maintained in its target position via a connecting longitudinal catheter or wire element extending from the active expandable portion along the intraluminal segment and extraluminal segment to outside the patient's body for temporary fixation.

In a further preferred embodiment the catheter device is a flow directed balloon tipped vascular catheter, wherein the distal end of the intraluminal segment is comprised of a compliant balloon, wherein the balloon diameter is smaller than the surrounding vessel diameter, the balloon being non-obstructive to surrounding fluid flow, wherein portions of the intraluminal segment are expandable and interactive with elements of bodily fluids in the target area.

In yet another preferred embodiment the device comprises a catheter housing, such as a sheath or guiding catheter, through which the device is forwarded to the a target site through a luminal organ and which permits movability of the device and the housing catheter relative to each other.

In yet another preferred embodiment, the device is designed to be refillable with said at least one pharmaceutically active compound.

It particularly preferred that said refilling is a refilling from the outside of the location of the device or form the outside of the subject's body.

In a further particularly preferred embodiment said refilling is to be performed via a hypotube connection of the sector of the device which comprises the at least one pharmaceutically active compound with a proximal end of a refilling device.

In yet another preferred embodiment the device according to the present invention has one or more of the following properties: (i) it is freely positionable in a target vessel, preferably in a minimal invasive manner; (ii) it is retrievable, preferably by catheter means and/or in a minimal invasive manner; (ii) it is anchorable in a target vessel; (iv) it comprises active and expandable portions which are self-expandable; (v) it comprises expandable portions which are mechanically triggered, preferably by balloon inflation; (vi) is designed to fit into and be connected to a permanent device present in a target vessel as a shuttle docking to a receiving site.

In yet another embodiment said device is provided in a tubular, onion like, pearl-chain-like, or a birds-nest like shape, or in any mixture of these shapes.

In another preferred embodiment said tubular shape is provided by a memory shaped spiraling wire, which forms a tubular spiral.

In yet another preferred embodiment the memory shaped spiraling wire is modified into a single spiral-like wire structure, characterized by incomplete wire circles and an interdigiting structure.

In yet another preferred embodiment said onion like or pearl-chain-like shape is provided by an elastic memory shape meshwork.

It is further preferred that the device is composed of, is partially composed of, or comprises structural support material selected from the group comprising (i) metal, such as stainless steel, gold, titanium, gold-titanium alloy, cobalt-chromium alloy, tantalum, platinum-radium alloy, tantalum alloy, magnesium, nickel-titanium alloy, e.g. nitinol, silver or copper, (ii) plastic or polymeric material, (iii) elastic memory shape meshwork material such as memory shape elastic wires, (iv) bactericidal material, or (v) a material readable by tomography or other imaging techniques such as X ray.

In a further particularly preferred embodiment said device is self-expandable.

In yet another preferred embodiment at least a portion of the device can be activated by balloon inflation.

In a further preferred embodiment the device comprises at least one docking element at the proximal end for retrieval.

In yet another preferred embodiment the device comprises a radiopaque marker.

In another preferred embodiment said radiopaque marker is located at the proximal and distal end, or on at least two opposite portions of the outermost structural element of the device body, allowing to judge radial expansion under medical imaging.

In a further embodiment said expandable portion is composed of elastic, foldable polymer material such as polyurethane, or of micromeshes comprising ultrathin wires, metallic or polymeric material.

In yet another preferred embodiment at least one cross-sectional area of the device body is at least partially covered by said expandable portion.

In yet another preferred embodiment the plane of at least one expandable portion is arranged perpendicular to the direction of the longitudinal axis of the device body, allowing for an increased exposure of the expandable portion to the flow of a bodily fluid

It is further envisaged by the present invention that the device additionally comprises one or more chemical and/or biological agents capable of binding a bacterial toxin and thereby removing said bacterial toxin from circulation, wherein said chemical and/or biological agents are preferably positioned on a membrane as defined herein above.

In a preferred embodiment said bacterial toxin is selected from the group of bacterial toxins comprising AB toxin, Adenylate cyclase toxin, Alpha toxin, Anthrax toxin, Botulinum toxin of type A, B, C, D, E, F or G, Cereulide, Cholera toxin, Cholesterol-dependent cytolysin, Clostridial Cytotoxin, Clostridium difficile toxin A, Clostridium difficile toxin B, Clostridium enterotoxin, Clostridium perfringens alpha toxin, Clostridium perfringens beta toxin, Coronatine, Cryptophycin, Delta endotoxin, Diphtheria toxin, Enterotoxin type B, Erythrogenic toxin, Exfoliatin, Heat-stable enterotoxin, Hemolysin of type E, alpha, beta or gamma, HrpZ, Leukocidin, Lipopolysaccharide such as Lipid A, Listeriolysin O, Microcin, Panton-Valentine leucocidin, Phenol-soluble modulin, Pneumolysin, Pseudomonas exotoxin, Pyocyanin, Rhs toxins, RTX toxin, Sakacin, Shiga toxin, Staphylococcus aureus alpha toxin, Staphylococcus aureus beta toxin, Staphylococcus aureus delta toxin, Streptolysin, Symplocamide A, Tabtoxin, Tetanolysin, Tetanospasmin, Tolaasin, Toxic shock syndrome toxin, Toxoflavin, Tracheal cytotoxin or Vibriocin.

It is further preferred that said binding to a bacterial toxin occurs via a protein, sugar- or liposaccharide-or lipopolysaccharide structure at the membrane of the device.

In a further preferred embodiment said protein-structure is a specific antibody, or fragment thereof, a peptide such as an antimicrobial peptide (AMP), an LPS-binding protein (LBP) or a lectin.

In yet another preferred embodiment said chemical and/or biological agent is linked to the device and/or a coating of the device via a spacer element, preferably a peptide or polypeptide, preferably the Fc part of an antibody or multi-histidine tag; a nucleic acid; a modified nucleic acid; or a polymer such as PEG, PLA, PVA, polyethylene, sphingomyelin or polypropylene.

In yet another preferred embodiment said spacer has a length of about 1 to 20 nm.

In an additional embodiment said spacer elements are provided in a density of 2 to 500 per μm² on the surface of the device.

It is further preferred that said chemical and/or biological agent comprises, essentially consists of, or consists of a binding domain capable of binding to a bacterial toxin.

In yet another preferred embodiment said binding domain has a length of about 20 to about 250 amino acids, preferably of about 20 to about 120 amino acids.

In a further preferred embodiment said binding domain has a length of 20, 25, 30, 35, 40, 45, 50, 55, 60, 65, 70, 75, 80, 85, 90, 95, 100, 105, 110, 115 or 120 amino acids.

In another aspect the present invention relates to a catheter device as defined herein above for use in treating a slow-healing or non-healing wound, diabetic foot, infections with multi resistant bacteria such as MRSA, or intoxication with bacterial toxins.

In a further aspect the present invention relates to a catheter device as defined herein above for use in preventing a slow-healing or non-healing wound, diabetic foot or intoxication with bacterial toxins, or for treating or preventing bacterial or virus infections and/or medical complications during transplantation, preferably skin transplantation.

In a preferred embodiment said (i) bacterial infection is an infection with a bacterium of the genus of Acinetobacter, Klebsiella, Pseudomonas, Escherichia, Enterobacter, Enterococcus, Staphylococcus, or Streptococcus; or (ii) said virus infection is an infection with a Coronavirus, preferably SARS-CoV2, HAV, HBV, HCV, or RSV.

In a further specific embodiment said bacterial infection is associated with additional conditions or diseases such as diabetes or gangrene.

In yet another preferred embodiment said device is designed to be implanted into a blood vessel such as an artery, an elastic artery, a distributing artery, an arteriole, a capillary, a venule or a vein.

It is further preferred that said device is implanted in a blood or lymphatic vessel upstream or downstream of a bacterial infection site in a subject.

According to a further preferred embodiment said device is implanted in close proximity to said bacterial infection site.

In yet another preferred embodiment the device is implanted in a blood vessel upstream of a tissue with a high risk of developing a bacterial infection.

In a further preferred embodiment the device is implanted during and/or after the treatment of a subject with a therapeutic agent.

In yet another preferred embodiment said treatment is an antibiotics therapy, preferably a systemic antibiotics therapy, and/or a pro-angiogenetic therapy.

In a further particularly preferred embodiment said device is implanted into a subject being at risk of developing a bacterial or virus infection or sepsis, or of developing a slow-healing or non-healing wound, or a diabetic foot.

In a further aspect the present invention relates to a method of treating a slow-healing or non-healing wound, diabetic foot, infections with multi resistant bacteria such as MRSA, a bacterial or virus infection, or intoxication with bacterial toxins, optionally associated with additional conditions or diseases such as diabetes or gangrene comprising implanting a catheter device as defined herein above into a subject in need thereof.

In yet another aspect the present invention relates to a method of preventing a slow-healing or non-healing wound, diabetic foot or intoxication with bacterial toxins, or for preventing bacterial or virus infections and/or medical complications during transplantation, preferably skin transplantation, optionally associated with additional conditions or diseases such as diabetes, gangrene, comprising implanting a catheter device as defined herein above into a healthy subject or a subject being at risk of developing a low-healing or non-healing wound, diabetic foot, or of becoming intoxicated with bacterial toxins.

In yet another aspect the present invention relates to a method of refilling a catheter device as defined herein above with at least one pharmaceutically active compound, preferably a pharmaceutically active compound as defined herein above

In a preferred embodiment said refilling is performed from the outside of the location of the device or form the outside of the subject's body.

In yet another preferred embodiment said refilling is performed via a hypotube connection of the sector of the device which comprises the at least one pharmaceutically active compound with a proximal end of a refilling device.

### BRIEF DESCRIPTION OF THE FIGURES

FIGS. 1 to 5 show embodiments of the present invention.

FIG. 6 shows an implant comprising three onion-like device bodies in tandem positions.

FIG. 7 shows an implant comprising a pearl-chain-like device body with differential pearl size diameters.

FIG. 8 shows an implant comprising a pearl-chain-like device body with tandem filters with different membrane locations.

FIG. 9A and B show different orientations of a filter membrane vis-à-vis the longitudinal axis of the implant body.

FIG. 10 shows an implant wherein cross-sectional planes of the circular loops or ellipsoids are oriented parallel to each other or may be oriented in an angle to each other.

FIG. 11 shows a Filterwire (Boston Scientific) device comprising filters resembling umbrellas on a wire with uniform pores of 80 um-110 um in diameter.

FIG. 12 shows an implant comprising a retrievable embolic filter.

FIG. 13 shows a bioactive coated implant comprising a microfilament assembly. The assembly is shown in 3 different versions, i.e. an open, closed and spiral version (1, 2 and 3). The figure further indicates the application of the catheter for placement and retrieval of the assembly.

FIG. 14 depicts the human artery system.

FIGS. 15 to 17 show examples of clinical uses of the filter device assembly implant according to the present invention.

FIG. 18 shows an example of a flow directional catheter device with an interactive expandable segment (balloon) according to the invention.

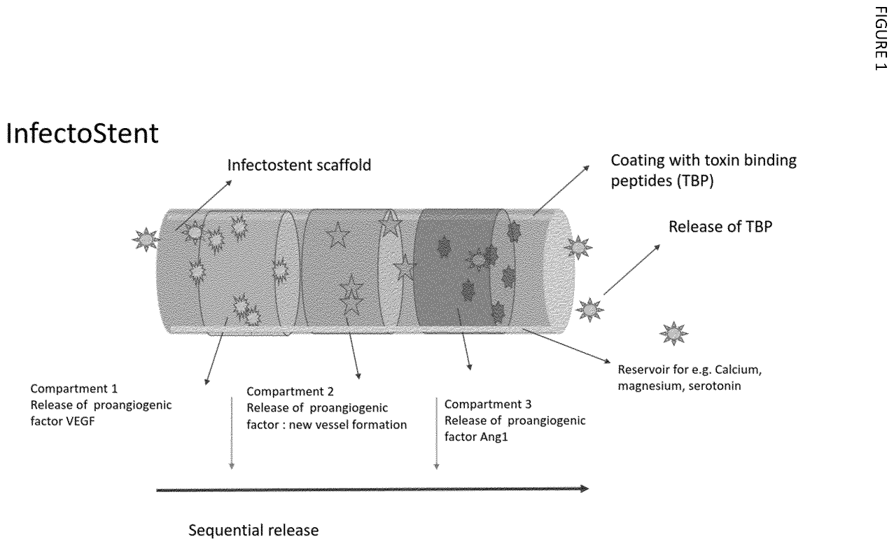

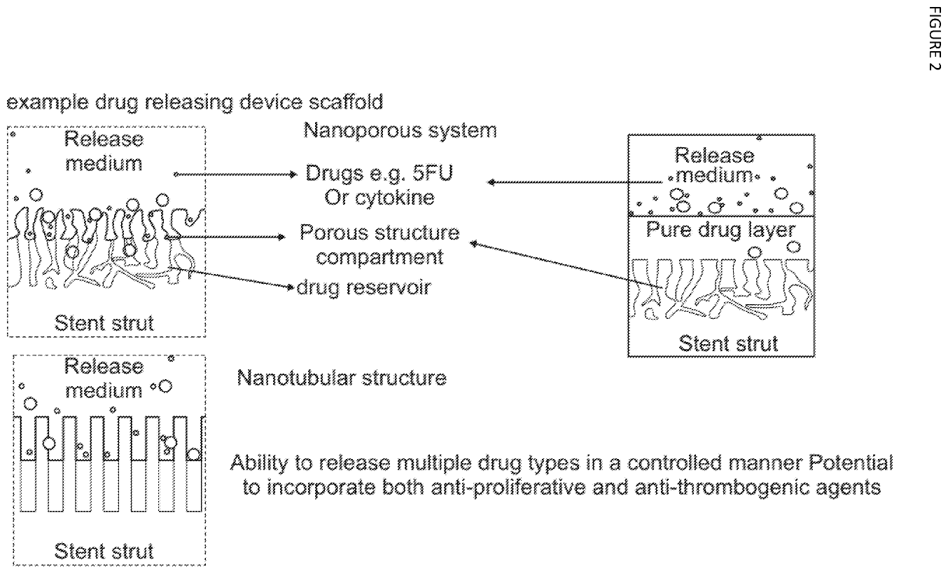

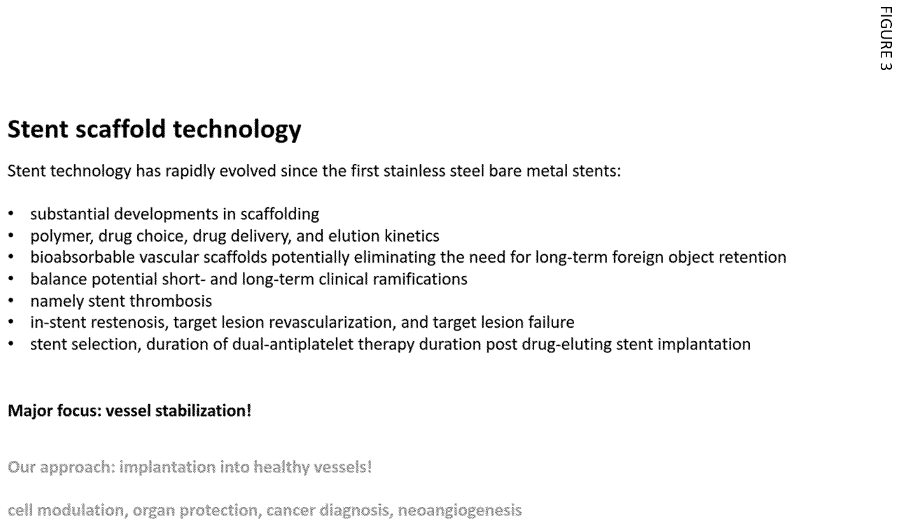

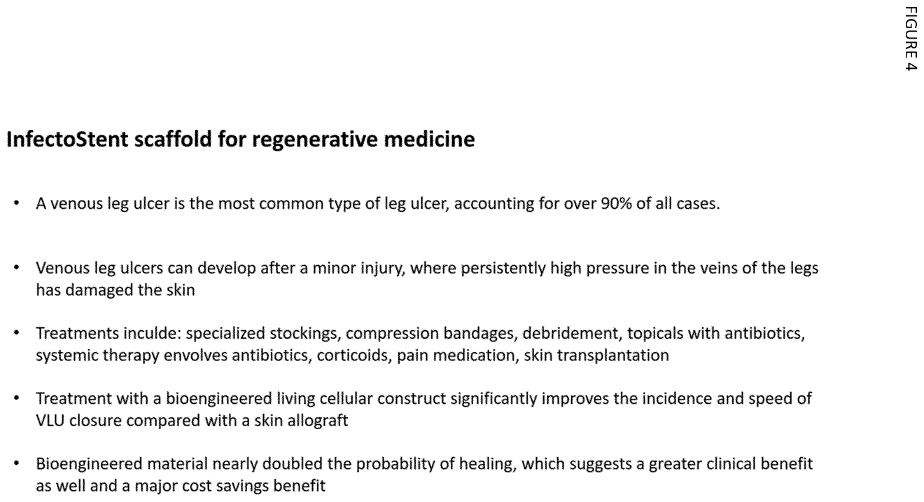

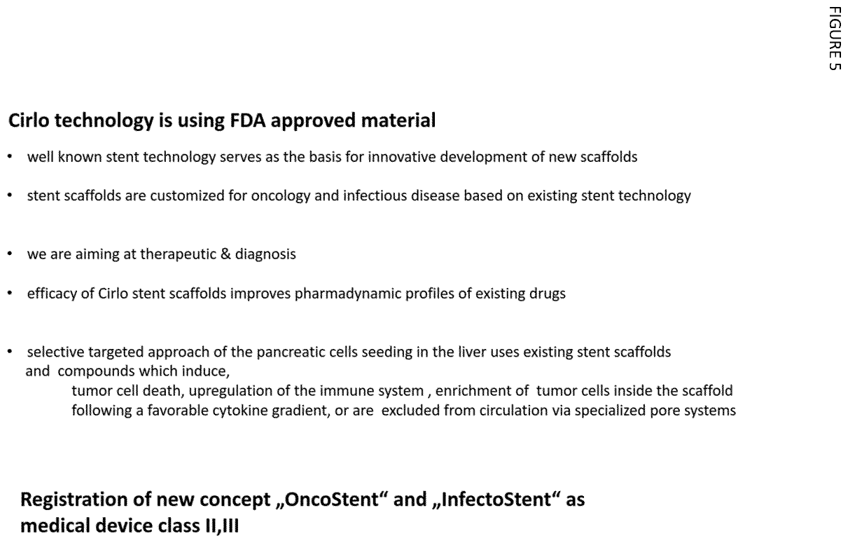

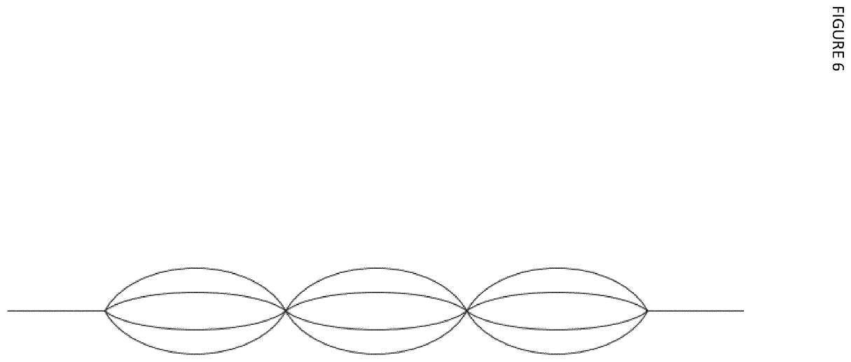

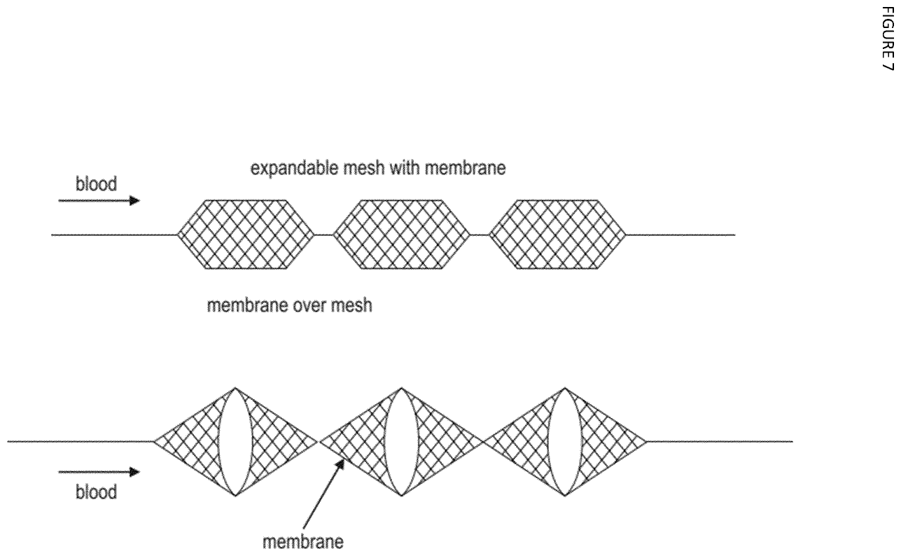

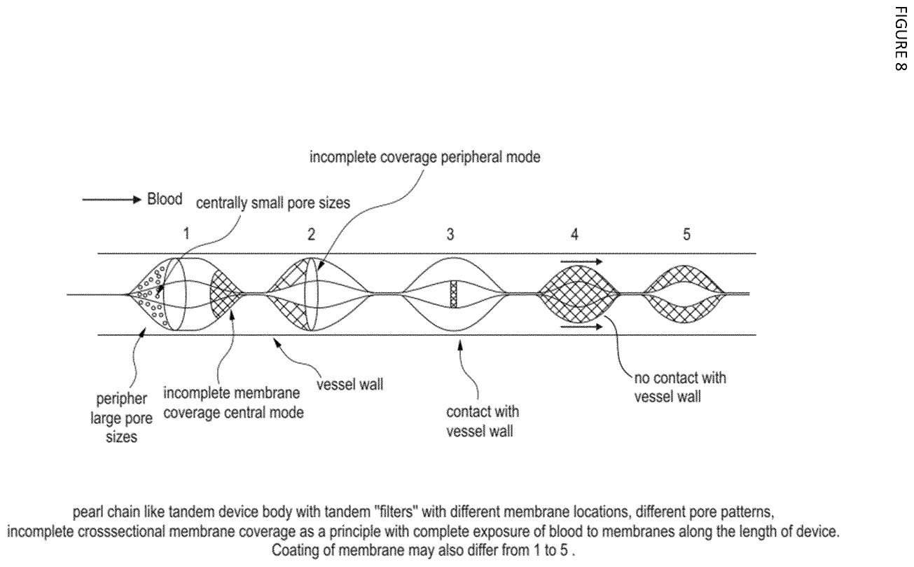

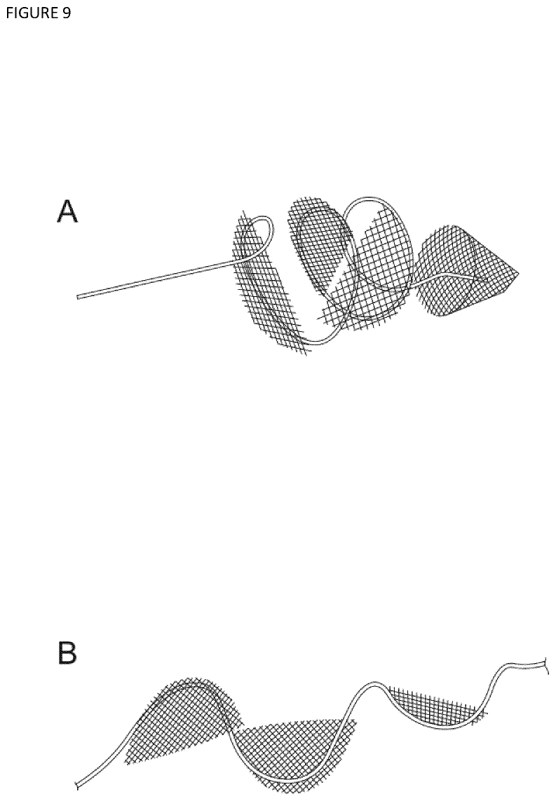

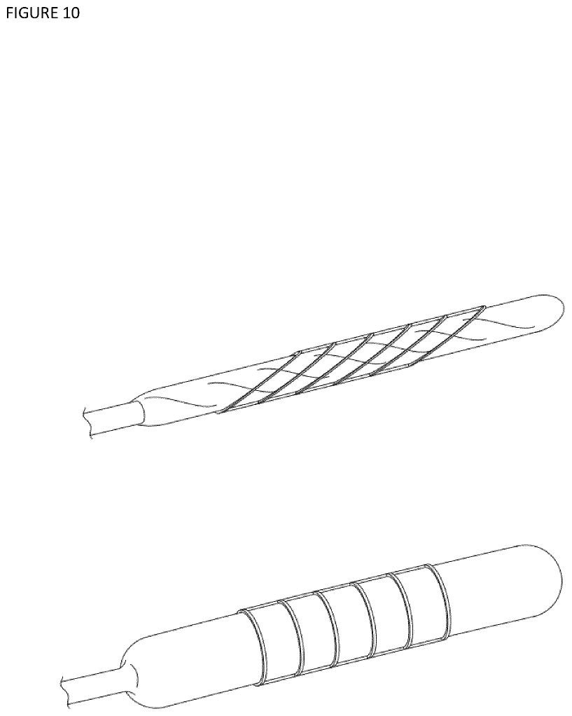

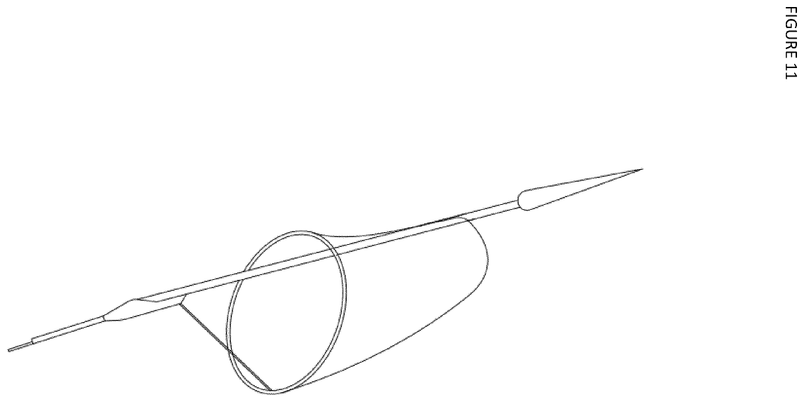

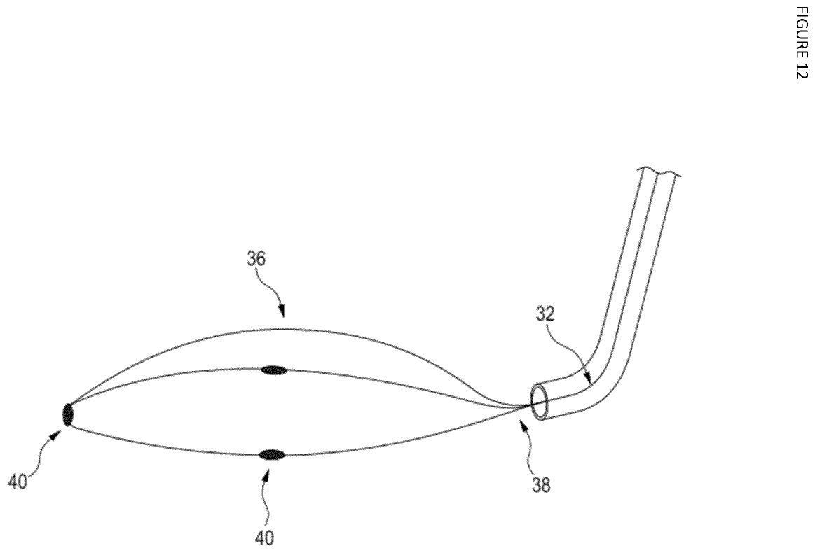

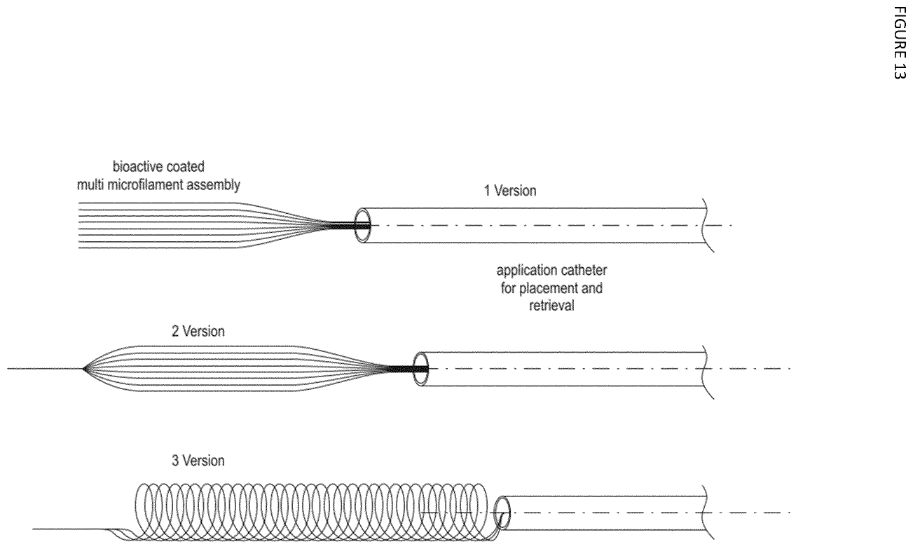

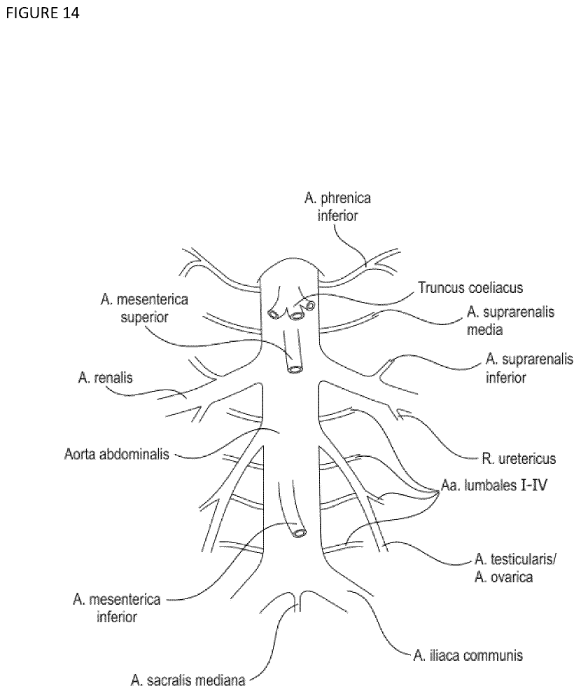

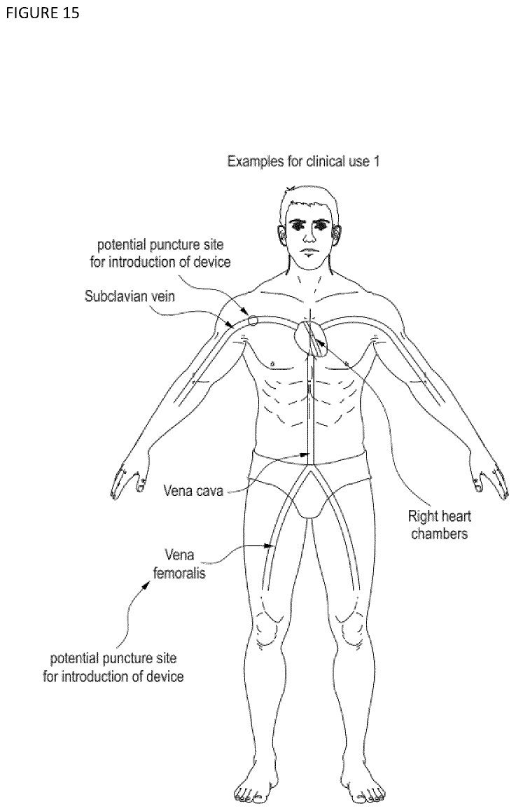

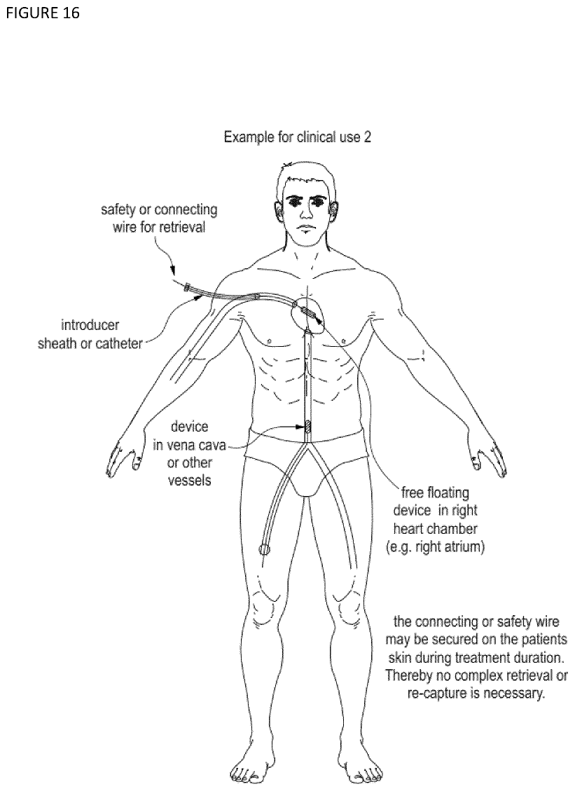

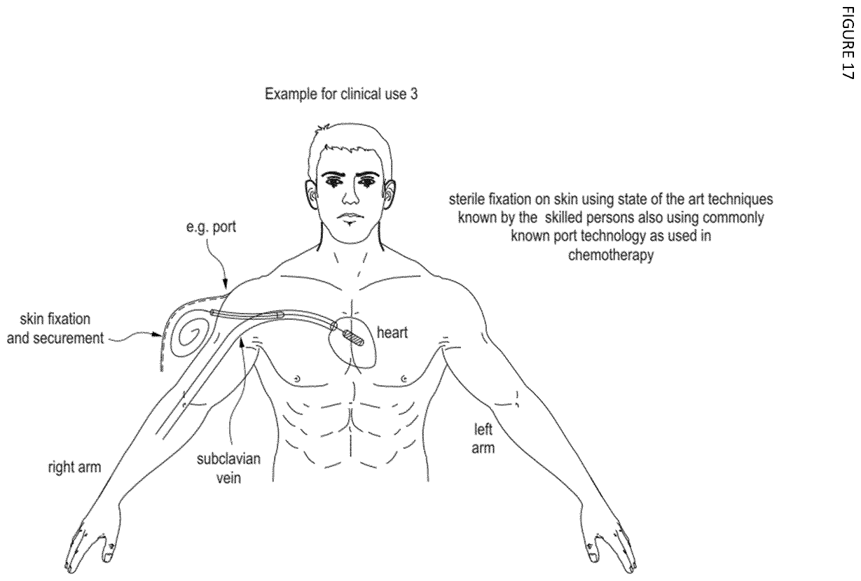

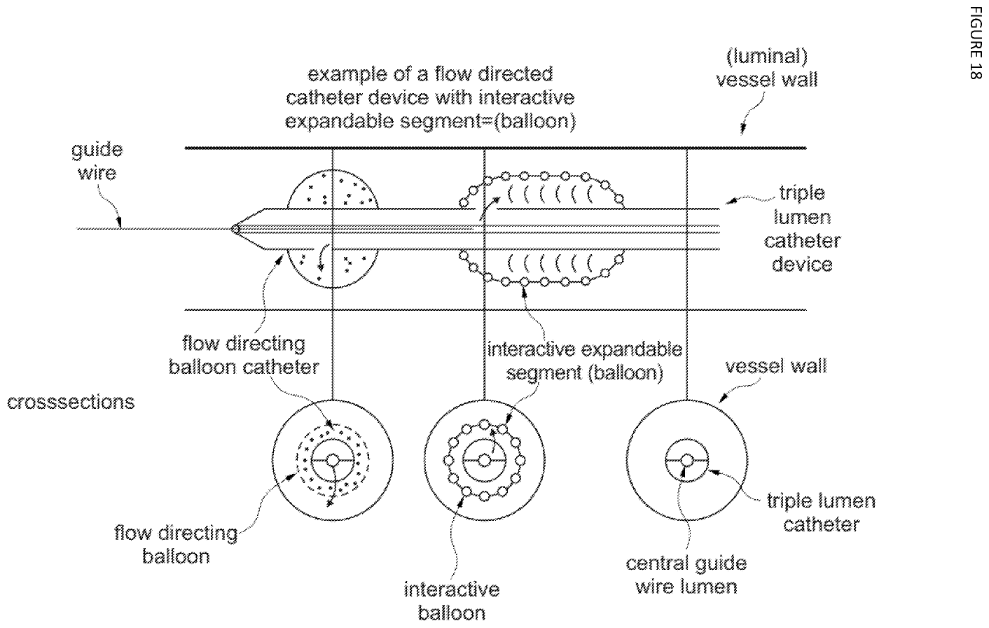

### DETAILED DESCRIPTION OF THE INVENTION

Although the present invention will be described with respect to particular embodiments, this description is not to be construed in a limiting sense.

Before describing in detail exemplary embodiments of the present invention, definitions important for understanding the present invention are given.

As used in this specification and in the appended claims, the singular forms of “a” and “an” also include the respective plurals unless the context clearly dictates otherwise

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” or “essentially consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “(i)”, “(ii)”, “(iii)” or “(a)”, “(b)”, “(c)”, “(d)”, or “first”, “second”, “third” etc. and the like in the description or in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms relate to steps of a method or use there is no time or time interval coherence between the steps, i.e. the steps may be carried out simultaneously or there may be time intervals of seconds, minutes, hours, days, weeks etc. between such steps, unless otherwise indicated.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As has been set out above, the present invention concerns in one aspect a catheter device comprising at least one pharmaceutically active compound, wherein the device is capable of releasing said pharmaceutically active compound over an extended period.

The term “catheter device” as used herein refers to simple device being placed in an intraluminal site, e.g. of an organ or in any other part of the vascular system. Preferably, the catheter device device is a tubular and longitudinally extending device assembly with an intraluminal and an extraluminal segment and a proximal and a distal end. The intraluminal segment, in further embodiments, comprises at least one reversibly expandable portion. The extension of the expandable portion may depend on the envisaged use and place. In a preferred embodiment the expandable portion extends at least 2 mm longitudinally, e.g. 2 mm, 2.5 mm, 3 mm, 4 mm, 5 mm etc.

In central embodiment of the present invention the expandable portion is permeable for a bodily fluid. The term includes, for example, lymphatic fluids and preferably blood.

It is further preferred that the expandable portion is capable of increasing the surface area of the intraluminal segment. Such an increase can be, for example, at least a two fold over the length of the expandable portion. Further envisaged are 2.5×, 3×, 4× or 5× increases

According to the present invention the pharmaceutically active compound comprised within the device is an angiogenesis promoting factor and/or an inhibitor of an angiogenesis inhibiting factor.

An “angiogenesis promoting factor” can, for example, be a promoting chemokine, a fibroblast growth factor, hepatocyte growth factor (HGF), a hypoxia-inducible factor, a platelet-derived growth factor, a transforming growth factor beta, or a vascular endothelial growth factor.

Envisaged examples of promoting chemokine are CXC-1, CXC-2, CXC-3, CXC-5, CXC-6, CXC-7, and CXC-8.

Envisaged examples of fibroblast growth factors are FGF-1 and FGF-2.

Envisaged examples of hypoxia-inducible factors are HIF-1, HIF-2 and HIF-3.

Envisaged examples of platelet-derived growth factor are PDGF-A, PDGF-B, PDGF-C and PDGF-D.

Envisaged examples of transforming growth factor beta are TGFbeta-1, TGFbeta-2 and TGFbeta-3.

Envisaged examples of vascular endothelial growth factors are VEGF-A, VEGF-B, VEGF-C, VEGF-D and PIGF.

An “inhibitor of an angiogenesis inhibiting factor” as used in the contex of the present invention is an angiopoietin, angiostatin, an inhibiting chemokine, endostatin, an interferon, pigment epithelium-derived factor (PEDF) or a thrombospondin.

Envisaged examples of angiopoietins are Ang-1 and Ang-2.

Envisaged examples of inhibiting chemokine are CXC-4, CXC-9, CXC-10, CXC-11, CXC-12, and CXC-14.

Envisaged examples interferons are INF-alpha, INF-beta and INF-gamma.

Envisaged examples of thrombospondins are TSP-1, TSP-2, TSP-3, TSP-4 and TSP-5.

According to specific embodiments the pharmaceutically active compounds present in or on the device may be provided in any combination or amount suitable for the intended treatment purpose.

The device may, in particularly preferred embodiments, additionally comprise a further pharmaceutically active compound of the group of antibiotics, anticoagulants and/or analgesics and/or serotonin and/or divalent ions such as Ca²⁺ or Mg²⁺.

The term “antibiotic” as used herein relates to an antimicrobial substance active against bacteria, i.e. antibacterial agent for fighting bacterial infections. Antibiotics either kill or inhibit the growth of bacteria. A limited number of antibiotics also possess antiprotozoal activity. Antibiotics typically have different forms and structures. The present invention explicitly also refers to antibiotics which have not yet been discovered. According to preferred embodiments, the antibiotics may be a penicillin or related compound, e.g. amoxicillin, ampicillin, oxacillin or dicloxacillin; or a tetracycline or related compound such as demeclocycline, doxycycline, eravacycline, minocycline or omadacycline. Also envisaged are cephalosporins, e.g. cefaclor, cefotaxime, ceftazidime, or cefuroxime. Also envisaged are quinolones such as ciprofloxacin, levofloxacin or moxifloxacin. Further preferred are lincomycins such as clindamycin, or lincomycin. The present invention also relates to sulphonamides such as sulfamethoxazole, trimethoprim or sulfasalazine. Further envisaged is the use of glycopeptide antibiotics. Examples of this family are dalbavancin, ortavancin, telavancin or vancomycin. Further preferred are aminoglycosides such as gentamicin, tobramycin, or amikacin. Also envisaged are ansamycines such as rifampicin, rifamycin B, rifamycin SV, rifabutin, rifapentine, or rifamixin. A further envisaged antibiotic is fosfomycin, fusidic acid and linezolid. In another embodiment, the antibotioc is a carbapenem such as imipenem, cilastatin, merpoenem, dorpenem or ertapenem. Also the use of minocylin is envisaged.

The term “anticoagulant” as used herein refers to a chemical substance that prevents or reduces coagulation of blood, prolonging the clotting time. Envisaged examples of anticoagulants include coumarins such as warfarin, heparins such as low weight heparin, fondaparinux, idraparinux or idrabiotaparinux, edoxaban, betrixaban, dabigatran, rivaroxaban, apixaban, or hementin.

The term “analgesic” as used herein refers to a group of drugs used to achieve analgesia. The analgesics act in various ways on the peripheral and central nervous system. Envisaged examples of analgesics include paracetamol, salicylates, and opioid drugs such as morphine and oxycodone.

In particularly preferred embodiment of the invention the device comprises the pharmaceutically active compound(s) in one or more compartments. The “compartment” as used herein refers to a separate section or part of the catheter device, wherein pharmaceutically active compounds of a defined loading volume may be kept isolated from other compounds or parts of the catheter. In one specific embodiment, the loading volume may be in the rage from 0.1 mL to 10 mL. The compartments may be used as storage areas for the pharmaceutically active compounds to be released over an extended period of time in a controlled manner. The wording “capable of releasing the pharmaceutically active compound(s) over an extended period” means capable of releasing the pharmaceutically active compound(s) at therapeutically effective rates for an extended period of time. In one embodiment the extended period is a period of about 7 days to 3 months, for example about 7 days to 2 months, about 7 days to 1 month, about 7 days to 25 days, about 7 days to 20 days, about 7 days to 15 days, about 7 days to 10 days, or any value between the above mentioned.

It is preferred that the compartments are capable of releasing the pharmaceutically active compound(s) over an extended period and can be controlled externally. Accordingly, in some embodiments, the release maybe controlled externally via a handheld device. The term “handheld device” refers to a portable, electronic device, which is supplied with power via an accumulator or a rechargeable battery. Such handheld devices may comprise a pocket computer, a tablet, a smartphone, a laptop computer, a personal digital assistant (PDA), and the like.

In another particularly preferred embodiment the compartments is provided in the at least one expandable portion of the device. It is particularly preferred to be liquid permeable and capable of releasing said pharmaceutically active compound(s) into bodily fluids or target tissues.

In specific embodiments, the compartment is a drug containing surface area or macro-reservoir. The reservoir can comprise mono- or multi-regional reservoir compartments which are adapted to provide controlled and variable delivery or release of two or more pharmaceutically active compounds in a simultaneous or sequential manner. Said macro-reservoirs may be in the form of a tube, pore, pouch, balloon, and the like, and can further comprise a cover or sealing to prevent the active compounds from an uncontrolled leaking out of the compartment. The cover may be for example a non-porous septum or membrane, or a membrane which dissolves or degrades over time, or a membrane capable of altering its permeability by varying external stimuli, but is not limited thereto. For example, the membranes can also contain an ordered array of stimuli-responsive core-shell type polystyrene latex particles, which change their size in response to external stimuli, acting as “on-off” switches or “permeability valves”, regulating the permeation through membrane channels. Further externally controlled stimuli may comprise heat, or electrical, magnetic or ultrasound stimuli.

In further embodiments, the drug containing surface area comprises a porous surface, or comprises one or more drug reservoirs such as micro-reservoirs.

The term “porous surface” as used herein means that the device comprises pores, preferably of different sizes, which are preferably further arranged in a membrane structure. These structures are, in further embodiments, provided at the surface of the expandable portions, allowing for a maximization of interaction with components of the bodily fluids. A “micro-reservoir” is a smaller version of the macro-reservoir, which is typically provided in the context of the porous surface as defined herein. It comprises one or more of the active compounds according to the present invention.

In further embodiments the reservoir(s) is/are imbedded within a membrane, preferably a porous membrane, or within the porous surface. Accordingly, the device as defined above comprises at least filter membranes. The term “filter membrane” as used herein relates to a selective barrier, mainly performing the function of a separator, e.g. by allowing for a filtering process. Pores of the porous membranous may have a pore diameter which ranges from about 25 nm to about 100 μm in diameter.

In preferred embodiments, the pores have different size ranges includes the range of about 25 nm to 100 nm in diameter, the range of about 100 nm to 10 μm in diameter, the range of about 10 μm to 25 μm in diameter and the range of about 25 μm to 100 μm in diameter. These ranges, which may all be present in one device or only a sub-group thereof, are adapted to the intended use of the device, i.e. the compounds to be released, the interaction intended etc.

In further particularly preferred embodiments the present invention also relates to a compliance or noncompliant balloon, flow directing or provided as carrier/container of a pharmaceutically active compounds as defined herein. The balloon may accordingly be used as macroreservoir and release controlling system.

In further embodiments, the balloon is a porous balloon with single or multiple mini pores. This allows for the release of the pharmaceutically active compounds in a controllable manner, e.g. by control of pressure inside the balloon from outside. For example, the control may be provided by an operator or may be an automated pressure control

Further envisaged is a control by the viscosity of a fluid, e.g. a pharmaceutically active compound and e.g. contrast medium, which is typically used in interventional radiology. The contrast medium typically has a high viscosity, leakage through micropores is thus achieved in a predictable and controllable way with or without additional balloon pressure control.

In a particularly preferred embodiment the expandable portion is a porous balloon, e.g. comprising pores as defined herein. According to further embodiments, it is in its expanded state at least one third smaller in diameter than the diameter of the luminal target organ.

In further preferred embodiments the expandable portion is composed of elastic, foldable polymer material such as polyurethane, or of micromeshes comprising ultrathin wires, metallic or polymeric material.

It is particularly preferred that the porosity of the balloon permits selective fluid permeability. This advantageously permits interaction with surrounding environments by controlled release of pharmaceutically active compound(s) from the balloon and attraction of surrounding fluid elements to the balloon.

In further embodiments the longitudinal extension of the intraluminal device is in a tandem position and all expandable portions permit flow of a bodily fluid around or through the expandable portions. It is particularly preferred that the at least one expandable portion is in fluid connection via a tubular channel with the proximal end of the extraluminal segment.

Further envisaged are embodiments wherein at least one wire channel extending from the distal tip of the intraluminal segment leads through at least a portion of the extraluminal segment.

Also envisaged are device forms, wherein the expandable portions are a combination of a scaffold and balloon. The at least one expandable active portion may further be detachable for anchoring within the luminal target organ. A reversible le anchoring approach is also envisaged. In this context, the reversible anchoring mechanism is preferably comprised within the detachable device.

Also preferred are embodiments, wherein the active expandable portion is maintained in its target position via a connecting longitudinal catheter or wire element extending from the active expandable portion along the intraluminal segment and extraluminal segment to outside the patient's body for temporary fixation.

In another preferred embodiment the catheter device is a flow directed balloon tipped vascular catheter, wherein the distal end of the intraluminal segment is comprised of a compliant balloon, wherein the balloon diameter is smaller than the surrounding vessel diameter, the balloon being non-obstructive to surrounding fluid flow, wherein portions of the intraluminal segment are expandable and interactive with elements of bodily fluids in the target area.

All device forms as described herein comprise a catheterhousing, which for example comprises a sheath or guiding catheter. Through the catheterhousing the device can be forwarded to the target site, e.g. through a luminal organ. This approach permits movability of the device and the housing catheter relative to each other.

In a specific embodiment of the present invention, the device is designed to be refillable with at least one pharmaceutically active compound. The refilling may, for example, be a refilling from the outside of the location of the device or from the outside of the subject's body. The refilling process can be performed manually by using a syringe, or automatically by using a pump system. Typically, refilling is carried out via one or more catheter ports which are designed for the purpose of refilling or aspiration of a reservoir, or for catheter access.

In another specific embodiment, the refilling is to be performed via a hypotube connection of the sector of the device which comprises the at least one pharmaceutically active compound with a proximal end of a refilling device. The term “hypotube” refers to a long metal tube with micro-engineered features along its length which is a component of catheters, used in conjunction with balloons and stents to open up clogged arteries. Typically, the balloon portion of the catheter is attached to the head of the hypotube.

The device according to the present invention may be provided, in preferred embodiments, with one or more suitable properties, which can be combined or mixed according to necessities or circumstances. In certain embodiments, all properties may be given in a device. The device according to the invention is hence designed to comprise one or more of said properties, in particular of properties (i) to (vi) as mentioned below.

These properties include: (i) the device is freely positionable in a target vessel. The term “freely positionable” means that the device can be placed at any position in a target vessel. Potential size differences between target vessels may be reflected by using differently sized devices, or by making use of mechanisms which allow to adjust the size of the device to the target vessel, e.g. by further opening or closing the device. It is preferred that device is designed to be freely positionable in a minimal invasive surgery approach, e.g. with endoscopic technology including cameras and grapplers etc.

Furthermore, (ii) the device is retrievable. The term “retrievable” as used herein means that the device can removed from its implantation site, e.g. by reducing its size or retracting extended elements, without leaving significant residues and without destroying or damaging the target vessel where is has been implanted. The way the device is retrieved can be any mechanism known to the skilled person. Preferably, the retrieval is performed by catheter means. It is further preferred that the retrieval be performed in a minimal invasive manner, e.g. making use of catheters, endoscopic technology etc. It is particularly preferred that least one docking element is present at the proximal end of the device. The term “docking element” as used herein relates to a structural component which allows for an interaction with an auxiliary tool such as a catheter or endoscope tool etc. The interaction may, in particular, be designed for a retrieval of the device, e.g. after a certain period of time. The docking may, for example, include a mechanical coupling between the device and an interaction tool such as a catheter. Alternatively, the device may provide a docking element in the form of a protrusion which is easily reachable and detectable, allowing for a grabbing or catching of the device, e.g. in analogy to vena cava filters.

A further property (iii) of the device according to the present invention is that it is anchorable in a target vessel. The term “anchorable” as used herein means that the device cannot be moved or does not float within the target vessel where it has been implanted, but stays at the position of its implantation. This is preferably achieved by a contact between the expanded device and the surrounding tissue or vessel wall wherein the contact force is depending on the radial expansion force of the device or certain device elements, e.g. in analogy to self-expanding peripheral stents or vessel occluders. Also anchoring can be achieved by adding extensions of the device which seek contact or extrude into neighboring tissue or vessel walls as described for atrial occluders such as watchman occluders.

Yet another property (iv) it comprises active and expandable portions which are self expandable.

Furthermore, (v) it comprises expandable portions which are mechanically triggered, preferably by balloon inflation.

In addition, (vi) the device is designed to fit into a permanent implant present in a target vessel. The device may, according to this property, be designed as a moveable and retrievable part of an implant. The implant may be present at a certain position in a target vessel. The device according to the present invention may be introducible into said implant and, e.g. after a certain period of time or after having reached the end of its working period (e.g. after 2, 3, 4, 5, 6, 7, 8, 9, 10, 11, 12, 15, 18, 20, 24 or more months), be removed therefrom. The device according to the invention may hence, in a preferred embodiment, be designed as a shuttle entity, which temporarily docks at a permanent implant present in a target vessel. The shuttle may, in further embodiments, be retrievable with a catheter, more preferably in a minimal invasive manner.

In further specific embodiment, the device may also be catheter based. The term “catheter based” as used herein means that the device can be packed and/or provided to the patient inside of a catheter. The term “catheter” as used herein relates to a thin tube made from medical grade materials that can be inserted in the body to treat diseases or perform a surgical procedure. A catheter may be any suitable catheter known to the skilled person. Typical examples include polymer-based catheters comprising material such as silicone rubber, nylon, polyurethane, polyethylene terephthalate (PET), latex, or thermoplastic elastomers. Also envisaged are polyimidine catheters. The catheter may, in certain embodiments, be connected to a deployment mechanism and may house a medical device that can be delivered over a guidewire. The catheter may include a guidewire lumen for over-the-wire guidance and may be used for delivering a device according to the invention to the target vessel. In certain embodiments, the catheter may have braided metal strands within the catheter wall to increase structural integrity. The structural elements of the catheter tip may further be bonded or laser welded to the braided strands of the catheter to improve the performance characteristics of the catheter tip.

The present invention further envisages that the device is provided in any suitable form or architecture. It is particularly preferred that the device is provided in a tubular, onion like, pearl-chain-like, or a birds-nest like shape, or in any mixture of these shapes. Examples of corresponding forms and shapes can, for example, be derived from FIG. 24, 25, 26, 27, 28, 29, 30, or 31. The form or shape of the device may, in certain embodiments, be followed also be the form or shape of the filter membranes. For example, the device according to the present invention may comprise alternating non-completely covering filter membranes which are provided in a pearl-chain, onion type or birds-nest like shape. In other embodiments, the device comprises a multitude of longitudinally extending microfilaments, which may be arranged parallel or non parallel to each other in straight configuration or tortuous e.g. spiraling configurations.

The elongated tubular shape is preferably provided by a memory shaped wire, or memory shaped spiraling wire, which forms a tubular spiral. An example is a flexible nitinol wire. More preferably, the memory shaped spiraling wire may be modified into a single spiral-like wire structure, characterized by incomplete wire circles and an interdigiting structure.

In another specific embodiment the device may have a grid-net-like or scaffold-like shape. It may comprises one or more chemical and/or biological agents as defined herein. The device may further or alternatively comprise one or more drug containing surface areas or macro-reservoirs as described herein.

The device may be composed of, or be partially composed of, or comprise any suitable structural support material. According to certain embodiments, the structural support material may be metal. Preferred examples are stainless steel, gold, titanium, gold-titanium alloy, cobalt-chromium alloy, tantalum, tungsten, platinum-radium alloy, tantalum alloy, magnesium, nickel-titanium alloy, e.g. nitinol, silver or copper. Alternatively or additionally, the support material may be plastic or polymeric material. Also envisaged is the use of memory shape materials, e.g. elastic memory shape meshwork material. Preferred examples include memory shape elastic wires. In yet another alternative, the structural support material may be a material readable by tomography or other imaging techniques, e.g. X ray. In certain specific embodiments the device body may not be biodegradable or composed of biomaterial or biodegradable material. The term “device body” as used herein relates to all parts of the device besides the one or more chemical and/or biological agents as defined herein above. Typically, the device body relates to the structural components of the device which provide for suitable placement at the desired target vessel and its capability of resisting impacting surrounding forces such as blood flow, vessel contraction or the like.

In alternative embodiments, the device or the device body may be biodegradable or composed of biomaterial or of biodegradable material, e.g. the device is composed of, is partially composed of, or comprises structural support material selected from the group comprising biodegradable or bioresorbable material. In a group of embodiments, the device may be made of polylactic acid, which is a naturally dissolvable material that is used in medical implants such as dissolving sutures. In certain further embodiments, the flexible tube and the balloon as described herein may be removed after implanting the device. Accordingly, the present invention envisages a biodegradable device which provides a temporary mechanical scaffolding. Subsequently, it may be bioabsorbed within a reasonable period, leaving behind the healed and remodeled artery preventing possible complications with devices that have be recovered by surgery. It is particularly preferred that the material for the biodegradable device constitutes a reasonable combination of a technical suitable degradation rate and mechanical integrity. An overall time period for degradation of up to 3 years, e.g. of 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 2.5 years, 3 years or any time value in between the mentioned values is envisaged. In further embodiments, the device may comprise pharmaceutical compositions or compounds, e.g. one or more of those mentioned herein, which are released in accordance with the degree of disintegration or degradation of the device.

In accordance with certain specific embodiments of the present invention, the biodegradable device is manufactured with, or composed of at least two classes of materials: polymers and metals. For example, metals to be used in the context of biodegradable devices may be magnesium alloys, iron and iron alloys, or zinc and zinc alloys. Clinical studies have shown that magnesium is an excellent candidate for cardiovascular applications as Mg ions inhibit platelet activation, relax vascular smooth muscle cells, and prevent vasoconstriction and subsequent increase blood pressure by inhibiting hormones such as angiotensin and norepinephrine. Also magnesium, due to its high electronegativity, exhibits excellent hypothrombogenic property. Above all excess Mg from the human body is believed to be efficiently eliminated by the kidney. Certain Mg alloys such as Mg-2.2Nd-0.1Zn-0.4Zr (JDBM-2) show very high biocompatibility, mechanical integrity, and degradation kinetics and are preferred herein.

Further envisaged are nano polymeric foam blends in a solid. The present invention additionally envisages the use of a range of synthetic biodegradable polymers, based on PLA, PGA, or co-polymers (e.g., PLGA) thereof, which may be provided in several morphologies and architectures. Also envisaged are different bioactive ceramics, such as calcium phosphates, hydroxyapatite powders and bioactive glass fibers, and as well as highly porous biocompatible nanocomposites. With wishing to be bound by theory, it is believed that the rate of scaffold bioactivity can be controlled by the amount of bioactive filler incorporated in the polymer matrix.

Further envisaged polymeric materials are poly-L-lactic acid (PLLA), polyglycolic acid (PGA), poly(D,L-lactide/glycolide) copolymer (PDLA) and polycaprolactone (PCL). Examples of suitable and envisaged resorbable polymers include PL Poly(L-lactide), PC Poly(ϵ-caprolactone), PLC Poly(L-lactide/ϵ-caprolactone), PLG Poly(L-lactide/Glycolide), PDL Poly(DL-lactide), PL Poly(L-lactide), PC Poly(ϵ-caprolactone), PLC Poly(L-lactide/ϵ-caprolactone), PLG Poly(L-lactide/Glycolide), PDL Poly(DL-lactide), PLDL Poly(L-DL lactide), PG Poly(Glycolide), PLDL Poly(L-DL lactide) and PG Poly(Glycolide).

The present invention also envisages that device is self-expandable. The term “self-expandable” as used herein relates to the property of the device to be expandable within a target vessel once it has reached its envisaged destination. The self-expansion may, for example, be started when withdrawing auxiliary tools such as catheters, as defined herein above. The self-expansion of the device may, in preferred embodiments, be activated by balloon inflation. It is preferred that at least a portion of the device, or the entire device be activated by balloon inflation. The term “balloon inflation” as used herein refers to the activity of expansion of a device by inflating a balloon element within the device. For example, a self-expanding device may comprise securing bands preventing the self-expansion of the device during the passage to the extended destination. Upon arrival, a balloon may be introduced into the device and be inflated within the device. Subsequently, said securing bands may be broken which leads to a self-expansion of the device. Subsequently, the balloon may be withdrawn, e.g. via catheter element. In a specific embodiment the device further comprises a docking element at the proximal end for retrieval.

In further embodiments, the device comprises one more than one localization markers. These markers should preferably be opposite to each other. The term “localization marker” as used herein means that device comprises an element which can be detected from the outside, e.g. during surgery or when monitoring the device during operation. It is preferred to use a radiopaque marker, preferably located at the proximal and distal end, or on at least two opposite portions of the outermost structural element of the device body, allowing to judge radial expansion under medical imaging. The term “radiopaque marker” as used herein relates to materials which block X-ray radiation. Examples of such markers include platinum, gold, tantalum, or stainless steel, or a radiopaque ink. It is particularly preferred that the radiopaque marker is included in the device design. For example, the marker may be located at the proximal and distal end, or on at least two opposite portions of the outermost structural element of the device body. Such a design can advantageously allow to judge radial expansion under medical imaging, e.g. online X-ray analysis, and thus improve device expansion and placing etc.

In a further embodiment at least one cross-sectional area of the device body is at least partially covered by said expandable. In yet another preferred embodiment the plane of at least one expandable portion is arranged perpendicular to the direction of the longitudinal axis of the device body, allowing for an increased exposure of the membrane to the flow of a bodily fluid.

Also envisaged is a device assembly which is connected to an energy emanating and/or producing module, which allows to destroy bacteria or similar target cells. The energy emanating and/or producing module may, for example, generate light, an electrical field, a magnetic field, heat or ultrasound, or any combination or sub-group of the above. The energy emanating module may preferably be situated outside of the subject's body and be connected therewith via a cable, endoscopic device, optical fiber, tube or any other suitable means. It is preferred that a device which is designed to make use of light energy comprises one or more optical fibers for light application. The term “optical fiber” as used herein relates to a flexible, transparent fiber made of silica or plastic for transmitting light between the two ends of the fiber widely used in fiber-optic communications. The optical fiber is hence meant to be a light transmitting element. In the context of the present embodiments it is envisaged to allow the application of light at one or more defined sites of the device and/or close to the device, preferably at a site where target cells, e.g. bacteria, are accumulated or are present. The term “light application”as used herein refers to any type of photo or light conveyed induction or usage in the context of the device as described herein. The device according to this aspect of the invention may accordingly be connected to a light emanating entity, which is preferably connected to an optical fiber. The light may be monochromatic light or a laser beam. The light application may be performed at different wavelengths, or ranges of wavelengths. Typically, a range between 350 nm to 750 nm is used. Particularly preferred wavelengths are 375-400 nm, 630 nm, 635 nm, 652 nm, 665 nm, 670 m, 689 nm, and 732 nm. Preferably, the light application is used for photodynamic therapy. This therapeutic approach, which is envisaged by the present invention, requires the presence of photosensitizer drugs, which may be placed on the device or associated to it. Suitable and envisaged examples of photosensitizer drugs are photofrin, verteporfin, foscan, levulan, metivix, benzvix, hexvix, purlytin, BOPP, photochlor, lutex, Pc4, or talaporfin. The application of light, e.g. of a wavelength as mentioned above, which is advantageously adapted or adaptable to the photosensitizer drug used activates the photosensitizer which lead to type 1 or type 2 reactions and ultimately transform or destroy tissue or cells, e.g. via reactive oxygen species or via radicals. Further details would be known to the skilled person or can be derived from suitable literature sources such as Dolmans et al., 2003, Nature Reviews, Cancer, 3, 380-387.

It is particularly preferred that said light application is used in the context of bacteria, which can be destroyed, e.g. multi resistant bacteria such as MRSA. In certain embodiments photosensitizer drugs may be provided independently form the outside at the site of the device, or they may be present in a reservoir structure of the device as defined herein and can be released deliberately.

In certain embodiments it is envisaged to provide a slow release of drugs or pharmaceutical compounds, preferably photosensitizers as defined above. Preferably, a slow release of photosensitizers as defined above, e.g. photosensitizers specific for certain bacteria such as MRSA is envisaged in order to prevent bacterially or virally caused diseases, preferably severe bacterially or virally caused diseases, preferably sepsis, more preferably severe sepsis. It is accordingly envisaged to target bacteria via chemical specification of photosensitizers which incorporate into the bacteria, e.g. bacteria which are present in the blood stream. The light induction as mentioned above will lead to the destruction of bacteria containing the photosensitizers. Advantageously, the bacterial load inside critically ill patients, preferably with large wounds, can be reduced with the described approach. According to specific embodiments, a slow release of pharmaceutical compounds, pharmaceutical compositions, preferably of photosensitizers is implemented with a specific coating of the device. For example, the coating may be a multilayer coating, which releases pharmaceutical compounds, e.g. photosensitizers. The release may take place over weeks, months or years.

In further embodiments the device may be used in the context of an electrical field or a magnetic field. This field may preferably be provided from the outside of the subject via a suitable field generator. The field may preferably be focused to the site of the device and accordingly be used to destroy bacteria, which can be destroyed, e.g. multi resistant bacteria such as MRSA at said site. Also envisaged is the use of heat, which may be applied to the site of the device via focused infrared treatment. The use of ultrasound may accordingly be focused to the device and be implemented by a transducer which is placed at the skin of the subject in the vicinity of the device. Further details would be known to the skilled person or can be derived from suitable literature sources such as Sengupta and Balla, 2018, Journal of Advanced Research, 14, 97-111.

In a further embodiment said device additionally comprises one or more chemical and/or biological agents capable of binding a bacterial toxin and thereby removing said bacterial toxin from circulation. Biological agents capable of binding a bacterial toxin comprise but are not limited to antibodies, complement, mannose-binding lectin (MBL), opsonins, lectins, peptidoglycan-binding proteins, receptors such as toll like receptors, and the like. Of particular interest are endogenous glycan sequences expressed at the surface of cells, particularly epithelial cells that represent the principal binding sites for pathogens at the initiation of a host tropism type infection. Further examples include, but are not limited to glycoproteins (eukaryotic glycoproteins, proteoglycans, glycomucins), and glycolipids, including natural, synthetic, and recombinant versions thereof as well as homologues, analogues, and functional equivalents thereto. These same glycan signatures may also constitute “self-antigens” that can be used to mask the foreign nature of the chemical and/or biological agents, thereby providing them with reduced immunogenicity. The glycan signatures can be either harvested directly from host tissues of interest or derived therefrom, using phage display and recombinant techniques.

In a preferred embodiment, the chemical and/or biological agents are positioned on a membrane as defined herein. The term “positioned on a membrane” refers in general to the immobilization or incorporation of said chemical and/or biological agents on a membrane of the catheter device. In the case of antibody immobilization, current procedures utilize conventional molecular conjugation techniques, which are based on conjugation of molecules using chemical binding techniques such as with biotinylation or binding with various polymers, often block copolymers. Common techniques for positioning or immobilization of chemical and/or biological agents include, but are not limited to reduction amination, diazo coupling, use of isothiocyanates, amidation, use of hom-bifunctional reagents, cycloadditions, maleimide addition, thioether linkages, oxime conjugation, stauding ligation olefin metathesis, biotinylation and PEGylation. Typically, bloc copolymers are often used to facilitate such conjugations. Molecules of interest can also be incorporated directly into a material matrix which constitutes the body of the substrate or which is applied as a coating on a substrate.

In one embodiment, the bacterial toxin is selected from the group of bacterial toxins comprising AB toxin, Adenylate cyclase toxin, Alpha toxin, Anthrax toxin, Botulinum toxin of type A, B, C, D, E, F or G, Cereulide, Cholera toxin, Cholesterol-dependent cytolysin, Clostridial Cytotoxin, Clostridium difficile toxin A, Clostridium difficile toxin B, Clostridium enterotoxin, Clostridium perfringens alpha toxin, Clostridium perfringens beta toxin, Coronatine, Cryptophycin, Delta endotoxin, Diphtheria toxin, Enterotoxin type B, Erythrogenic toxin, Exfoliatin, Heat-stable enterotoxin, Hemolysin of type E, alpha, beta or gamma, HrpZ, Leukocidin, Lipopolysaccharide such as Lipid A, Listeriolysin O, Microcin, Panton-Valentine leucocidin, Phenol-soluble modulin, Pneumolysin, Pseudomonas exotoxin, Pyocyanin, Rhs toxins, RTX toxin, Sakacin, Shiga toxin, Staphylococcus aureus alpha toxin, Staphylococcus aureus beta toxin, Staphylococcus aureus delta toxin, Streptolysin, Symplocamide A, Tabtoxin, Tetanolysin, Tetanospasmin, Tolaasin, Toxic shock syndrome toxin, Toxoflavin, Tracheal cytotoxin or Vibriocin.

In a further embodiment said binding to a bacterial toxin occurs via a protein-, sugar- or liposaccharide-or lipopolysaccharide structure at the membrane of the device. The term “lipopolysaccharide” (LPS) or “lipoglycans” or “endotoxins” refers to large molecules that are the major components of the outer membrane of gram-negative bacteria, contribute greatly to the structural integrity of bacteria, and protect the bacterial membrane. Typically, LPS consists of a lipid and a polysaccharide composed of O-antigen, outer core and inner core joined by a covalent bond.

It is further preferred that the protein-structure is a specific antibody, or a fragment thereof, a peptide such as an antimicrobial peptide (AMP) an LPS-binding protein (LBP) or a lectin. The term “LPS-binding protein” or “LBP” refers to a protein that is capable of binding to bacterial lipopolysaccharide (LPS) to elicit immune response by presenting the LPS to important cell surface pattern recognition receptors (CD14 and TLR4).

In a further embodiment the chemical and/or biological agent as defined herein is linked to the device and/or a coating of the device via a spacer element. The term “spacer element” as used herein relates to a distance piece which is capable of spatially separating the biological agent form the surface of the device. This separating allows for a sterically unhindered interaction of the biological agent with a component of the bodily fluid. In preferred embodiments the spacer elements are composed or partially composed of a peptide or a polypeptide. It is particularly preferred that the Fc part of an antibody or multi-histidine tag be used. Also envisaged is the employment of a nucleic acid; a modified nucleic acid; or a polymer such as PEG, PLA, PVA, polyethylene, sphingomyelin or polypropylene. The spacer element may have any suitable length. In a preferred embodiment, the spacer element has a length of about 1 to 20 nm, e.g. 1, 2, 3, 4, 5, 6, 7, 8, 9, 10, 11, 12, 13, 14, 15, 16, 17, 18, 19 or 20 nm or any value in between the mentioned values. Also envisaged are shorter spacer elements in a length of 7 to 25 amino acids, e.g. 7, 8, 9, 10, 11, 12, 13, 14, 15, 16, 17, 18, 19, 20, 21, 22, 23, 24, 25 amino acids, or equivalents thereof.

The present invention further relates to biological agents—as part of the device—which comprise, essentially consists of, or consists of a binding domain capable of binding to a bacterial toxin. The term “binding domain capable of binding to a bacterial toxin” as used herein relates to an amino acid sequence which comprises a non-covalent binding functionality for a bacterial toxin as mentioned herein. Preferably, the binding domain is capable of binding to comprising AB toxin, Adenylate cyclase toxin, Alpha toxin, Anthrax toxin, Botulinum toxin of type A, B, C, D, E, F or G, Cereulide, Cholera toxin, Cholesterol-dependent cytolysin, Clostridial Cytotoxin, Clostridium difficile toxin A, Clostridium difficile toxin B, Clostridium enterotoxin, Clostridium perfringens alpha toxin, Clostridium perfringens beta toxin, Coronatine, Cryptophycin, Delta endotoxin, Diphtheria toxin, Enterotoxin type B, Erythrogenic toxin, Exfoliatin, Heat-stable enterotoxin, Hemolysin of type E, alpha, beta or gamma, HrpZ, Leukocidin, Lipopolysaccharide such as Lipid A, Listeriolysin O, Microcin, Panton-Valentine leucocidin, Phenol-soluble modulin, Pneumolysin, Pseudomonas exotoxin, Pyocyanin, Rhs toxins, RTX toxin, Sakacin, Shiga toxin, Staphylococcus aureus alpha toxin, Staphylococcus aureus beta toxin, Staphylococcus aureus delta toxin, Streptolysin, Symplocamide A, Tabtoxin, Tetanolysin, Tetanospasmin, Tolaasin, Toxic shock syndrome toxin, Toxoflavin, Tracheal cytotoxin or Vibriocin.

In further aspect, the present invention relates to a device which comprises one or more elements or components which are capable of interacting with a virus, e.g. as defined herein, preferably capable of binding said virus and capturing or trapping it, e.g. from a bodily fluid or the ciruculation. Corresponding elements may be human ACE2 receptor, or a sub-portion thereof, a human ACE2 (hACE2) protein or a truncated version thereof, or a sub-portion thereof, representing the BD (binding domain), or Coronaviridae spike, preferably a SARS-CoV-2 spike protein or a sub-portion thereof or any derivatives of any of the above, a proprotein convertase furin. The term “sub-portion”as used herein relates to a fragment or truncated version of the protein, which keeps or still shows the relevant function of the parental molecule. Such a function may be a biochemical or enzymatic function, or an immunological function, e.g. showing a binding effect or the like.

Further envisaged are antibodies against one or more viruses as described herein. It is generally envisaged that the device for treating or preventing viral infections comprises biocoating molecules which are based on the proteins necessary for pathogen/virus to attach to the host cell and/or cell entry. The mimicry of the host cell by displaying the truncated host entry mechanism proteins will trap the virus inside the device and thus reduce the virus load from the circulation.

One aspect of the present invention relates to the device as described herein for use in treating or preventing a bacterial or virus infection, preferably of a slow-healing or non-healing wound, diabetic foot, infections with multi resistant bacteria such as MRSA, or intoxication with bacterial toxins. In a preferred embodiment it the device is for use in preventing bacterial or virus infections and/or medical complications during transplantation, preferably skin transplantation.

A “wound” as used herein refers a damaged area of the body that typically involves a laceration or breaking of a membrane such as the skin, and usually damage to underlying tissues. Many wounds pose no challenge to the body's innate ability to heal; some wounds, however, may not heal easily either because of the severity of the wounds themselves or because of the poor state of health of the individual. Wounds that exhibit improper or impaired healing, including delayed acute wounds and chronic wounds, generally have failed to progress through the normal stages of healing are referred to as “slow-healing wounds”. Typically, any wound that does not heal within a few weeks can be referred to as “slow-healing” or “non-healing wound”.

The term “diabetic foot” refers to a foot that exhibits any pathology that results directly from diabetes mellitus or any long-term complication of diabetes mellitus. Typically, the symptoms of a diabetic foot include infection, diabetic foot ulcer and neuropathic osteoarthrophathy.

The term “bacterial infection” encompasses superficial colonisation by bacteria, e.g. of the skin, intraauricular or mucosa as well as systemic infections, including infections of blood, tissues and organs. Also encompassed are infections of the gastrointestinal tract. The mucosal diseases can be caused by oral or vaginal infections. The oral or vaginal infections are for example the consequence of AIDS, chemotherapy or an immune suppressive therapy or immune suppressive conditions. The phrase “infections with multi resistant bacteria such as MRSA” refers to infections that are caused by a group of multiple drug resistant microorganisms such as methicillin-resistant Staphylococcus aureus (MRSA), a group of gram-positive bacteria that are genetically distinct from other strains of Staphylococcus aureus.

The term “intoxication with bacterial toxins” refers to the invasion of bacterial toxins into the human body through different ways that cause adverse reactions. “Intoxication”may for example to food-borne diseases that result from the spoilage of contaminated food.

The term “virus infection” as used herein encompasses viral infection of certain tissues, e.g. of the respiratory tract, the brain, the gastrointestinal tract, the liver or the mucosa as well as systemic infections, including infections of general tissues and organs.

In specific embodiments, the present invention envisages the use of a device as described herein for the treatment of critically ill patients, e.g. for the treatment of critically ill patients being afflicted by a bacterial or virus infection. The device according to the present invention may, in certain embodiments, be capable of capturing, for example, virus particles. For example, the device may comprise surface structures which are able to bind a virus or virion or a part of a virus. Such surface structures are preferably proteins or glycoproteins or sugar-comprising components. These structures may, in preferred embodiments, be placed in filter pores as described herein.

In a further embodiment, the bacterial infection is an infection with a bacterium of the genus of Acinetobacter, Klebsiella, Pseudomonas, Escherichia, Enterobacter, Enterococcus, Staphylococcus, or Streptococcus.

The infection may, for example, be an infection caused by a bacterium selected from Acinetobacter baumanii, Acinetobacter baylyi, Acinetobacter beijerinckii, Acinetobacter calcoaceticus, Acinetobacter gerneri, Acinetobacter haemolytica, Acinetobacter johnsonii, Acinetobacter junii, Acinetobacter Iwoffii, Acinetobacter twoneri, Acinetobacter vivanii, Klebsiella alba, Klebsiella aerogenes, Klebsiella granulomatis, Klebsiella grimontii, Klebsiella michiganensis, Klebsiella oxytoca, Klebsiella pneumoniae species such as Klebsiella pneumoniae ozaenae, Klebsiella pneumoniae pneumoniae, Klebsiella pneumoniae rhinoscleromatis, Klebsiella quasipneunominae, Klebsiella singaprensis, Klebsiella variicola, Pseudomonas aeruginosa, Pseudomonas chlororaphis, Pseudomonas fluorescens, Pseudomonas pertucinogena, Pseudomonas putida, Pseudomonas stutzeri, Pseudomonas syringae, Enterococcus alcedinis, Enterococcus aquimarinus, Enterococcus acini, Enterococcus durans, Enterococcus faecalis, Enterococcus faecium, Enterococcus gallinarum, Enterococcus haemoperoxidus, Enterococcus hirae, Enterococcus malodoratus, Enterococcus moraviensis, Enterococcus mundii, Enterococcus pseudoavium, Enterococcus raffinosus, Enterococcus solitaries, Escherichia coli, Escherichia albertii, Escherichia fergusonii, Escherichia hermanni, Escherichia vulneris, Staphylococcus argenteus, Staphylococcus arlettae, Staphylococcus aureus, Staphylococcus agnetis, Staphylococcus auricularis, Staphylococcus capitis, Staphylococcus caprae, Staphylococcus carnosus, Staphylococcus chromogenes, Staphylococcus cohnii, Staphylococcus condiment, Staphylococcus devriesei, Staphylococcus epidermidis, Staphylococcus fleurettii, Staphylococcus gallinarum, Staphylococcus haemolytica, Staphylococcus hominis, Staphylococcus hyicus, Staphylococcus intermedius, Staphylococcus kloosii, Staphylococcus lentus, Staphylococcus lugdunensis, Staphylococcus lutrae, Staphylococcus massiliensis, Staphylococcus microti, Staphylococcus muscae, Staphylococcus nepalensis, Staphylococcus pettenkoferi, Staphylococcus piscifermentans, Staphylococcus pseudintermedius, Staphylococcus pulvereri, Staphylococcus rostri, Staphylococcus saccharolyticus, Staphylococcus saprophytics, Staphylococcus schleiferi, Staphylococcus sciuri, Staphylococcus simiae, Staphylococcus simulans, Staphylococcus stepanovicii, Staphylococcus succinus, Staphylococcus vitulinus, Staphylococcus warneri, Staphylococcus xylosus, Streptococcus agalactiae, Streptococcus anginosus, Streptococcus constellatus, Streptococcus downei, Streptococcus dysglacticae, Streptococcus gordonii, Streptococcus infantarius, Streptococcus iniae, Streptococcus intermedius, Streptococcus lactarius, Streptococcus mitis, Streptococcus mutans, Streptococcus oralis, Streptococcus pneumoniae, Streptococcus pyogenes, Streptococcus peroris, Streptococcus salivarius, Streptococcus sanguinis, Streptococcus suis, Streptococcus sobrinus, Streptococcus thermophiles, or Streptococcus uberis, but is not limited thereto.

In certain embodiments the virus infection may be provoked or induced by any virus attacking a subject, e.g. a mammal, in particular a human being. The virus infection may, for example, be caused by a DNA or RNA virus. The DNA virus may be a dsDNA virus. The dsDNA virus may, in further embodiments, belong to the order of Caudovirales, Herpesvirales or Ligamenvirales, or belongs to the family of Adenoviridae, Ampullaviridae, Ascoviridae, Asfarviridae, Baculoviridae, Bicaudaviridae, Clavaviridae, Corticoviridae, Fuselloviridae, Globuloviridae, Guttaviridae, Hytrosaviridae, Iridoviridae,

Lavidaviridae, Marseilleviridae, Mimiviridae, Nimaviridae, Nudiviridae, Pandoraviridae, Papillomaviridae, Phycodnaviridae, Plasmaviridae, Polydnaviruses, Polyomaviridae, Poxviridae, Sphaerolipoviridae, Tectiviridae, Tristromaviridae or Turriviridae. Preferably, it is a human papillomavirus (HPV), a herpes virus, or an adenovirus.

In a further embodiment the virus infection to be treated according to the present invention may, for example, be caused by a ssDNA virus. The ssDNA virus may, in further embodiments, belong to the family of Anelloviridae, Bacilladnaviridae, Bidnaviridae, Circoviridae, Geminiviridae, Genomoviridae, Inoviridae, Microviridae, Nanoviridae, Parvoviridae, Smacoviridae or Spiraviridae.

In a further embodiment the virus infection to be treated according to the present invention may, for example, be caused by a dsRNA virus. The dsDNA virus may, in further embodiments, belong to the family of Amalgaviridae, Birnaviridae, Chrysoviridae, Cystoviridae, Endornaviridae, Hypoviridae, Megabirnaviridae, Partitiviridae, Picobirnaviridae, Reoviridae, Totiviridae, Quadriviridae, Botybirnavirus. Preferably, it is a rotavirus.

In a further embodiment the virus infection to be treated according to the present invention may, for example, be caused by a ssRNA virus. In specific embodiment, said virus may be a negative strand ssRNA virus. The negative strand ssRNA virus may, in further embodiments, belong to the order of Muvirales, Serpentovirales, Jingchuvirales, Mononegavirales, Goujianvirales, Bunyavirales or Articulavirales. In further preferred embodiments, the virus is a negative strand ssRNA virus belonging to the family of Filoviridae, Paramyxoviridae, Pneumoviridae or Orthomyxoviridae. In a particularly preferred embodiment, the virus is an RSV, metapneumovirus, or an influenza virus.

In a set of particularly preferred embodiments, the virus infection to be treated according to the present invention may, for example, be caused by a positive strand ssRNA virus. Said positive strand ssRNA virus may, for example, belong to the order of Nidovirales, Picornavirales or Tymovirales. In a preferred embodiment said virus is a positive strand ssRNA virus belonging to the family of Coronaviridae, Picornaviridae, Caliciviridae, Flaviviridae, Togaviridae. It is further preferred that virus is a rhinovirus, Norwalk-Virus, Echo-Virus or enterovirus. Further preferred is that the virus is HAV, HBV or HCV.

In a more preferred embodiment, the virus infection to be treated according to the present invention may, for example, be caused by a virus belonging to the family of Coronaviridae. Examples are Coronavirus or a member of the group of Coronaviruses. The group of Coronaviruses is typically divided into subgroups, i.e. alpha or beta coronaviruses. The present invention in very preferred embodiment, envisages the treatment of a virus infection caused by a human coronavirus or a Microchiroptera (bat) coronavirus or a coronavirus obtained from wild an animal belonging to the group of pangolins or similar animals or belonging the Pholidota group.

In a further preferred embodiment, the virus is a causative agent of a viral respiratory tract infection. Accordingly, the virus may belong to any of the above mentioned groups, families, classes or orders and be known to the skilled person as causing a viral respiratory tract infection.

In a particularly preferred embodiment said virus is a causative agent of MERS, SARS or COVID. In a more preferred embodiment, the virus is a causative agent of COVID-19, or a similar virally induced disease.

In a particularly preferred embodiment said virus is PHEV, FcoV, IBV, HCoV0C43 and HcoV HKU1, JHMV, HCoV NL63, HCoV 229E, TGEV, PEDV, FIPV, CCoV, MHV, BCoV, SARS-CoV, MERS-CoV or SARS-CoV-2, or any mutational derivative thereof. The term “mutational derivative thereof” as used herein relates to virus variants, which do not have the same genomic sequence as the mentioned viruses but is derived therefrom by mutational events which are typical for this virus group. These events may lead to changes in the infectious behavior of the virus, but still allows for a classification of the virus, thus identification of the virus as belonging to the group of coronaviruses.

In the most preferred embodiment said virus is SARS-CoV-2. In specifically preferred embodiment, the virus infection to be treated is or comprises a viral respiratory tract infection. It is particularly preferred that said virus infection is MERS, SARS or COVID, more preferably COVID-19.

In certain embodiments, said bacterial infection is associated with additional conditions or diseases such as diabetes or gangrene.

In further specific embodiments, the device of the present invention is specifically designed to be implanted into a blood vessel. Examples of envisaged blood vessels are an artery, an elastic artery, a distributing artery, an arteriole, a capillary, a venule or a vein. It is further envisaged to design the device for an implantation into the heart, preferably into a heart chamber or an elastic artery. The device may accordingly be designed as free floating device with connecting wires to maintain its position. This concept also allows for retrieval. In further specific embodiments, the device of the present invention is specifically designed to be implanted into a lymphatic vessel.

It is particularly preferred to that the device is implanted in a blood or lymphatic vessel upstream or downstream of a bacterial infection site in a subject. Alternatively, the device is implanted in close proximity to said bacterial infection site.

In further embodiments, the blood vessel is implanted upstream of a tissue with a high risk of developing a bacterial infection. The device may preferably be implanted during and/or after the treatment of a subject with a therapeutic agent. The treatment is, in typical embodiments, an antibiotics therapy, preferably a systemic antibiotics therapy, and/or a pro-angiogenetic therapy.

It is further preferred that the device is implanted into a subject being at risk of developing a bacterial or virus infection or sepsis, or of developing a slow-healing or non-healing wound, or a diabetic foot.

Also envisaged is a method of treating a slow-healing or non-healing wound, diabetic foot, infections with multi resistant bacteria such as MRSA, or intoxication with bacterial toxins, optionally associated with additional conditions or diseases such as diabetes or gangrene comprising implanting a catheter device as herein above into a subject in need thereof.

In a further aspect the invention relates to a method of preventing a slow-healing or non-healing wound, diabetic foot or intoxication with bacterial toxins, or for preventing bacterial infections and/or medical complications during transplantation, preferably skin transplantation, optionally associated with additional conditions or diseases such as diabetes, gangrene, comprising implanting a catheter device as defined herein above into a healthy subject or a subject being at risk of developing a low-healing or non-healing wound, diabetic foot, or of becoming intoxicated with bacterial toxins.

In another aspect the present invention relates to a method of capturing bacteria and/or viruses for a subsequent analysis, e.g. in an in vitro diagnostics approach. The method comprises a removal of the device comprising captured bacteria, preferably captured viruses and an assessment, e.g. with PCR techniques, mass spectroscopy or protein chemistry techniques or any other suitable approach. Further envisaged are quantitative measurements of viruses obtained with a method as described above.

In a final aspect the invention relates to a method of refilling a catheter device as defined herein above with at least one pharmaceutically active compound, preferably a pharmaceutically active compound as mentioned above. Said refilling is preferably performed from the outside of the location of the device or form the outside of the subject's bod. It is further preferred that said refilling is performed via a hypotube connection of the sector of the device which comprises the at least one pharmaceutically active compound with a proximal end of a refilling device.

The following figures are provided for illustrative purposes. It is thus understood that figures are not to be construed as limiting. The skilled person in the art will clearly be able to envisage further modifications of the principles laid out herein.

1. A catheter device comprising at least one pharmaceutically active compound, wherein the device is capable of releasing said pharmaceutically active compound over an extended period.

2. The catheter device of claim 1, wherein said pharmaceutically active compound is an angiogenesis promoting factor and/or an inhibitor of an angiogenesis inhibiting factor.

3. The catheter device of claim 2, wherein said angiogenesis promoting factor is a promoting chemokine, a fibroblast growth factor, hepatocyte growth factor (HGF), a hypoxia-inducible factor, a platelet-derived growth factor, a transforming growth factor beta, or a vascular endothelial growth factor.

4. The catheter device of claim 3, wherein said promoting chemokine is CXC-1, CXC-2, CXC-3, CXC-5, CXC-6, CXC-7, or CXC-8.

5. The catheter device of claim 3 or 4, wherein said fibroblast growth factor is FGF-1 or FGF-2.

6. The catheter device of any one of claims 3 to 5, wherein said hypoxia-inducible factor is HIF-1, HIF-2 or HIF-3.

7. The catheter device of any one of claims 3 to 6, wherein said platelet-derived growth factor is PDGF-A, PDGF-B, PDGF-C or PDGF-D.

8. The catheter device of any one of claims 3 to 7, wherein said transforming growth factor beta is TGFbeta-1, TGFbeta-2 or TGFbeta-3.

9. The catheter device of any one of claims 3 to 8, wherein said vascular endothelia) growth factor is VEGF-A, VEGF-B, VEGF-C, VEGF-D or PIGF.

10. The catheter device of any one of claims 2 to 9, wherein said angiogenesis inhibiting factor is an angiopoietin, angiostatin, an inhibiting chemokine, endostatin, an interferon, pigment epithelium-derived factor (PEDF) or a thrombospondin.

11. The catheter device of claim 10, wherein said angiopoietin is Ang-1 or Ang-2.

12. The catheter device of claim 10 or 11, wherein said inhibiting chemokine is CXC-4, CXC-9, CXC-10, CXC-11, CXC-12, or CXC-14.

13. The catheter device of any one of claims 10 to 12, wherein said interferon is INF-alpha, INF-beta or INF-gamma.

14. The catheter device of any one of claims 10 to 13, wherein said thrombospondin is TSP-1, TSP-2, TSP-3, TSP-4 or TSP-5.

15. The catheter device of any one of claims 1 to 14, wherein said device additionally comprises a further pharmaceutically active compound of the group of antibiotics, anticoagulants and/or analgesics and/or serotonin and/or divalent ions such as Ca²⁺ or Mg²⁺.

16. The catheter device of claim 15, wherein said antibiotic is penicillin such as amoxicillin, ampicillin, oxacillin or dicloxacillin; a tetracycline such as demeclocycline, doxycycline, eravacycline, minocycline or omadacycline; a cephalosporin such as cefaclor, cefotaxime, ceftazidime, cefuroxime; a quinolone such as ciprofloxacin, levofloxacin or moxifloxacin; a lincomycin such as clindamycin, or lincomycin; a sulphonamide such as sulfamethoxazole, trimethoprim or sulfasalazine; a glycopeptide antibiotic such as dalbavancin, ortavancin, telavancin or vancomycin; an aminoglycoside such as gentamicin, tobramycin, or amikacin; an ansamycine such as rifampicin, rifamycin B, rifamycin SV, rifabutin, rifapentine, or rifamixin; fosfomycin; fusidic acid; linezolid; or a carbapenem such as imipenem, cilastatin, merpoenem, dorpenem or ertapenem, or minocylin.

17. A catheter device comprising at least one elements which is capable of binding to or capturing a virus, preferably an ACE2 receptor or a sub-portion thereof, a human ACE2 (hACE2) protein or a truncated version thereof, or sub-portion thereof, a Coronaviridae spike protein such as a SARS-CoV-2 spike protein or a sub-portion thereof, or a proprotein convertase furin.

18. The catheter device of any one of claims 1 to 17, wherein the device is a tubular and longitudinally extending device assembly with an intraluminal and an extraluminal segment and a proximal and a distal end.

19. The catheter device of claim 18, wherein the intraluminal segment comprises at least one reversibly expandable portion.

20. The catheter device of claim 19, wherein the expandable portion extends at least 2 mm longitudinally.

21. The catheter device of any one of claims 19 to 20, wherein the expandable portion is permeable for a bodily fluid, preferably for blood.

22. The catheter device of any one of claims 19 to 21, wherein the expandable portion is capable of increasing the surface area of the intraluminal segment at least two fold over the length of the expandable portion.

23. The catheter device of any one of claims 1 to 22, wherein said device comprises the pharmaceutically active compound(s) in one or more compartments.

24. The catheter device of claim 23, wherein said compartments are capable of releasing said pharmaceutically active compound(s) over an extended period and/or can be controlled externally, preferably via a handheld device.

25. The catheter device of any one of claims 1 to 24, wherein said extended period is a period of about 7 days to 3 months.

26. The catheter device of any one of claims 23 to 25, wherein said compartments are provided in the at least one expandable portion, which is liquid permeable and capable of releasing said pharmaceutically active compound(s) into bodily fluids or target tissues.

27. The catheter device of any one of claims 18 to 26, wherein said compartment is a drug containing surface area or macro-reservoir.

28. The catheter device of claim 27, wherein said drug containing surface area comprises a porous surface, or comprises one or more drug reservoirs such as micro-reservoir.

29. The catheter device of claim 28, wherein the reservoir(s) is/are imbedded within a membrane, preferably a porous membrane, or within the porous surface.

30. The catheter device of any one of claims 19 to 29, wherein the at least one expandable portion is a porous balloon.

31. The catheter device of claim 30, wherein the porous balloon in its expanded state is at least one third smaller in diameter than the diameter of the luminal target organ.

32. The catheter device of claim 29 or 30, wherein the porosity of the balloon permits selective fluid permeability and wherein said permeability permits interaction with surrounding environments by controlled release of pharmaceutically active compound(s) from the balloon and attraction of surrounding fluid elements to the balloon.

33. The catheter device of any one of claims 19 to 32, wherein multiple expandable portions are arranged along the longitudinal extension of the intraluminal device in a tandem position and wherein all expandable portions permit flow of a bodily fluid around or through the expandable portions.

34. The catheter device of any one of claims 19 to 33, wherein the at least one expandable portion is in fluid connection via a tubular channel with the proximal end of the extraluminal segment.

35. The catheter device of any one of claims 19 to 34, wherein the longitudinally extending tubular device comprises at least one wire channel extending from the distal tip of the intraluminal segment through at least a portion of the extraluminal segment.

36. The catheter device of any one of claims 19 to 35, wherein the expandable portions are a combination of a scaffold and balloon.

37. The catheter device of any one of claims 19 to 36, wherein at least one expandable active portion is detachable for anchoring within the luminal target organ.

38. The catheter device of claim 37, wherein a reversible anchoring mechanism is comprised within the detachable device.

39. The catheter device of any one of claims 19 to 38, wherein the active expandable portion is maintained in its target position via a connecting longitudinal catheter or wire element extending from the active expandable portion along the intraluminal segment and extraluminal segment to outside the patient's body for temporary fixation.

40. The catheter device of any one of claims 19 to 39, wherein the catheter device is a flow directed balloon tipped vascular catheter, wherein the distal end of the intraluminal segment is comprised of a compliant balloon, wherein the balloon diameter is smaller than the surrounding vessel diameter, the balloon being non-obstructive to surrounding fluid flow, wherein portions of the intraluminal segment are expandable and interactive with elements of bodily fluids in the target area.

41. The catheter device of any one of claims 1 to 40 comprising of a catheter-housing, such as a sheath or guiding catheter, through which the device is forwarded to the a target site through a luminal organ and which permits movability of the device and the housing catheter relative to each other.

42. The catheter device of any one of claims 1 to 41, wherein the device is designed to be refillable with said at least one pharmaceutically active compound.

43. The catheter device of claim 42, wherein said refilling is a refilling from the outside of the location of the device or form the outside of the subject's body.

44. The catheter device of claim 42 or 43, wherein said refilling is to be performed via a hypotube connection of the sector of the device which comprises the at least one pharmaceutically active compound with a proximal end of a refilling device.

45. The catheter device of any one of claims 1 to 44, wherein said device has one or more of the following properties: (i) it is freely positionable in a target vessel, preferably in a minimal invasive manner; (ii) it is retrievable, preferably by catheter means and/or in a minimal invasive manner; (ii) it is anchorable in a target vessel; (iv) it comprises active and expandable portions which are selfexpandable; (v) it comprises expandable portions which are mechanically triggered, preferably by balloon inflation; (vi) is designed to fit into and be connected to a permanent device present in a target vessel as a shuttle docking to a receiving site.

46. The catheter device of any one of claims 1 to 45, wherein said device is provided in a tubular, onion like, pearl-chain-like, or a birds-nest like shape, or in any mixture of these shapes.

47. The catheter device of claim 46, wherein said tubular shape is provided by a memory shaped spiraling wire, which forms a tubular spiral.

48. The catheter device of claim 47, wherein the memory shaped spiraling wire is modified into a single spiral-like wire structure, characterized by incomplete wire circles and an interdigiting structure.

49. The catheter device of any one of claims 46 to 48, wherein said onion like or pearl-chain-like shape is provided by an elastic memory shape meshwork.

50. The catheter device of any one of claims 1 to 49, wherein said device is composed of, is partially composed of, or comprises structural support material selected from the group comprising (i) metal, such as stainless steel, gold, titanium, gold-titanium alloy, cobalt-chromium alloy, tantalum, platinum-radium alloy, tantalum alloy, magnesium, nickel-titanium alloy, e.g. nitinol, silver or copper, (ii) plastic or polymeric material, (iii) elastic memory shape meshwork material such as memory shape elastic wires, (iv) bactericidal material, or (v) a material readable by tomography or other imaging techniques such as X ray and wherein the device body.

51. The catheter device of any one of claims 1 to 50, wherein said device is self-expandable.

52. The catheter device of claim 51, wherein at least a portion of the device can be activated by balloon inflation.

53. The catheter device of any one of claims 45 to 52, wherein said device comprises at least one docking element at the proximal end for retrieval.

54. The catheter device of any one of claims 1 to 53, wherein said device comprises a radiopaque marker.

55. The catheter device of claim 54, wherein said radiopaque marker is located at the proximal and distal end, or on at least two opposite portions of the outermost structural element of the device body, allowing to judge radial expansion under medical imaging.

56. The catheter device of any one of claims 45 to 55, wherein said expandable portion is composed of elastic, foldable polymer material such as polyurethane, or of micromeshes comprising ultrathin wires, metallic or polymeric material.

57. The catheter device of any one of claims 45 to 56, wherein at least one cross-sectional area of the device body is at least partially covered by said expandable portion.

58. The catheter device of any one of claims 19 to 57, wherein the plane of at least one expandable portion is arranged perpendicular to the direction of the longitudinal axis of the device body, allowing for an increased exposure of the expendable portion to the flow of a bodily fluid.

59. The catheter device of any one of claims 1 to 16 or 18 to 58, wherein said device additionally comprises one or more chemical and/or biological agents capable of binding a bacterial toxin and thereby removing said bacterial toxin from circulation, wherein said chemical and/or biological agents are preferably positioned on a membrane as defined in any one of claims 29 to 58.

60. The catheter device of claim 59, wherein said bacterial toxin is selected from the group of bacterial toxins comprising AB toxin, Adenylate cyclase toxin, Alpha toxin, Anthrax toxin, Botulinum toxin of type A, B, C, D, E, F or G, Cereulide, Cholera toxin, Cholesterol-dependent cytolysin, Clostridial Cytotoxin, Clostridium difficile toxin A, Clostridium difficile toxin B, Clostridium enterotoxin, Clostridium perfringens alpha toxin, Clostridium perfringens beta toxin, Coronatine, Cryptophycin, Delta endotoxin, Diphtheria toxin, Enterotoxin type B, Erythrogenic toxin, Exfoliatin, Heat-stable enterotoxin, Hemolysin of type E, alpha, beta or gamma, HrpZ, Leukocidin, Lipopolysaccharide such as Lipid A, Listeriolysin O, Microcin, Panton-Valentine leucocidin, Phenol-soluble modulin, Pneumolysin, Pseudomonas exotoxin, Pyocyanin, Rhs toxins, RTX toxin, Sakacin, Shiga toxin, Staphylococcus aureus alpha toxin, Staphylococcus aureus beta toxin, Staphylococcus aureus delta toxin, Streptolysin, Symplocamide A, Tabtoxin, Tetanolysin, Tetanospasmin, Tolaasin, Toxic shock syndrome toxin, Toxoflavin, Tracheal cytotoxin or Vibriocin.

61. The catheter device of claim 59 or 60, wherein said binding to a bacterial toxin occurs via a protein-, sugar- or liposaccharide-or lipopolysaccharide structure at the membrane of the device.

62. The catheter device of claim 61, wherein said protein-structure is a specific antibody, or fragment thereof, a peptide such as an antimicrobial peptide (AMP), an LPS-binding protein (LBP) or a lectin.

63. The catheter device of any one claims 59 to 62, wherein said chemical and/or biological agent is linked to the device and/or a coating of the device via a spacer element, preferably a peptide or polypeptide, preferably the Fc part of an antibody or multi-histidine tag; a nucleic acid; a modified nucleic acid; or a polymer such as PEG, PLA, PVA, polyethylene, sphingomyelin or polypropylene.

64. The catheter device of claim 63, wherein said spacer has a length of about 1 to 20 nm.

65. The catheter device of claim 63 or 64, wherein said spacer elements are provided in a density of 2 to 500 per μm² on the surface of the device.

66. The catheter device of any one of claims 59 to 65, wherein said chemical and/or biological agent comprises, essentially consists of, or consists of a binding domain capable of binding to a bacterial toxin.

67. The catheter device of claim 66, wherein said binding domain has a length of about 20 to about 250 amino acids, preferably of about 20 to about 120 amino acids.

68. The catheter device of claim 67, wherein said binding domain has a length of 20, 25, 30, 35, 40, 45, 50, 55, 60, 65, 70, 75, 80, 85, 90, 95, 100, 105, 110, 115 or 120 amino acids.

69. A catheter device as defined in any one of claims 1 to 68 for use in treating a slow-healing or non-healing wound, diabetic foot, infections with multi resistant bacteria such as MRSA, bacterial or virus infections, or intoxication with bacterial toxins.

70. A catheter device as defined in any one of claims 1 to 68 for use in preventing a slow-healing or non-healing wound, diabetic foot or intoxication with bacterial toxins, or for preventing bacterial or virus infections and/or medical complications during transplantation, preferably skin transplantation.

71. The catheter device for use of claim 69 or 70, wherein (i) said bacterial infection is an infection with a bacterium of the genus of Acinetobacter, Klebsiella, Pseudomonas, Escherichia, Enterobacter, Enterococcus, Staphylococcus, or Streptococcus, or (ii) said virus infection is an infection with a Coroanvirus, preferably SARS-CoV2, HAV, HBV, HCV, or RSV.

72. The catheter device for use of any one of claims 69 to 71, wherein said bacterial infection is associated with additional conditions or diseases such as diabetes or gangrene.

73. The catheter device of any one of claims 1 to 68, or the catheter device for use of any one of claims 69 to 72, wherein said device is designed to be implanted into a blood vessel such as an artery, an elastic artery, a distributing artery, an arteriole, a capillary, a venule or a vein.

74. The catheter device of any one of claims 69 to 73, wherein said device is implanted in a blood or lymphatic vessel upstream or downstream of a bacterial infection site in a subject.

75. The catheter device for use of claim 74, wherein said device is implanted in close proximity to said bacterial infection site.

76. The catheter device of any one of claims 69 to 73, wherein said device is implanted in a blood vessel upstream of a tissue with a high risk of developing a bacterial infection.

77. The catheter device of any one of claims 69 to 73, wherein said device is implanted during and/or after the treatment of a subject with a therapeutic agent.

78. The catheter device of any one of claims 69 to 73, wherein said treatment is an antibiotics therapy, preferably a systemic antibiotics therapy, and/or a pro-angiogenetic therapy.

79. The catheter device of any one of claims 69 to 73, wherein said device is implanted into a subject being at risk of developing a bacterial or virus infection or sepsis, or of developing a slow-healing or non-healing wound, or a diabetic foot.

80. A method of treating a slow-healing or non-healing wound, diabetic foot, infections with multi resistant bacteria such as MRSA, a bacterial or virus infection, or intoxication with bacterial toxins, optionally associated with additional conditions or diseases such as diabetes or gangrene comprising implanting a catheter device as defined in any one of claims 1 to 68 into a subject in need thereof.

81. A method of preventing a slow-healing or non-healing wound, diabetic foot or intoxication with bacterial toxins, or for preventing bacterial or virus infections and/or medical complications during transplantation, preferably skin transplantation, optionally associated with additional conditions or diseases such as diabetes, gangrene, comprising implanting a catheter device as defined in any one of claims 1 to 68 into a healthy subject or a subject being at risk of developing a low-healing or non-healing wound, diabetic foot, or of becoming intoxicated with bacterial toxins.

82. A method of refilling a catheter device as defined in any one of claims 23 to 68 with at least one pharmaceutically active compound, preferably a pharmaceutically active compound as defined in any one of claims 2 to 17.

83. The method of claim 82, wherein said refilling is performed from the outside of the location of the device or form the outside of the subject's body.

84. The method of claim 82 or 83, wherein said refilling is performed via a hypotube connection of the sector of the device which comprises the at least one pharmaceutically active compound with a proximal end of a refilling device.
